(19) United States
(12) Patent Application Publication
(10) Pub. No.: US 2003/0144475 A1
(43) Pub. Date: Jul. 31, 2003

(54) Human protein and cdna

(76) Inventors: Seishi Kato, Kanagawa (JP); Chikashi Eguchi, Kanagawa (JP); Mihoro Saeki, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 09/890,688

(22) PCT Filed: Dec. 6, 2000
(86) PCT No.: PCT/JP00/08631

(30) Foreign Application Priority Data
Dec. 6, 1999 (JP) 11-346863
Dec. 6, 1999 (JP) 11-346864
Feb. 8, 2000 (JP) 2000-031062
Feb. 10, 2000 (JP) 2000-034091
Feb. 10, 2000 (JP) 2000-034090
Feb. 14, 2000 (JP) 2000-035829
Feb. 14, 2000 (JP) 2000-035899
Mar. 14, 2000 (JP) 2000-071161
May 30, 2000 (JP) 2000-160851

(51) Int. Cl. C07K 14/435; C07H 21/04

(52) U.S. Cl. 530/350; 536/23.1

(57) ABSTRACT

The present application provides a purified human protein, DNA fragment encoding the protein, expression vector for the DNA fragment, various cells transformed with the expression vector, and antibody against the protein. The purified protein in this invention is useful as a medicinal or as an antigen for manufacturing the antibody against the proteins. Further, the protein is useful as a search reagent for elucidating the intracellular protein network or as a protein source for screening such a protein as binding with a small molecule medicinal. The human cDNA of this invention is useful as a probe for gene diagnosis or as a gene source for gene therapy. Further, it can be also used as a gene source for mass production of the protein encoded by the cDNA. The expression vector being capable of translating in vitro or expressing the DNA within the host cell can be used for producing the human protein of this invention in vitro or within various host cells. The cells carrying the gene and expressing excessively it can be utilized for detecting the corresponding receptors and ligands or screening new small molecule medicinal or the like. The antibody against the protein of this invention can be used as a means for purifying the protein or for examining an expression level and localization site of the intracellular protein.

### TECHNICAL FIELD

The present invention relates to a purified human protein, DNA fragment encoding the protein, expression vector of the DNA fragment, various cells transformed with the expression vector, and antibody against the protein. The protein in this invention is useful as a medicinal or as an antigen for manufacturing an antibody against the protein. Further, the protein is useful as a search reagent for elucidating the intracellular protein network or as a protein source for screening such a protein as binding with a small molecule medicinal. The human cDNA of this invention is useful as a probe for gene diagnosis or as a gene source for gene therapy. Further, it can be also used as a gene source for mass production of the protein encoded by the cDNA. The expression vector can be used for producing the protein of this invention in vitro or within various host cells. The cell carrying these genes and expressing excessively them can be utilized for detecting the corresponding receptors and ligands or screening new small molecule medicinal or the like. The antibody against the protein of this invention can be used as a means for purifying the protein or for examining an expression level and localization site of the intracellular protein.

### BACKGROUND ART

Human proteins are essential components of the cells consisting of human bodies. Of them, there are (1) cytoskeleton proteins keeping the cell form or participating in the intracellular transportation of materials or cell movement, (2) metabolic enzymes participating in the intracellular metabolism of materials, (3) proteins concerning the energy production, (4) proteins transferring information on the cell proliferation and division, (5) translation-related proteins concerning the synthesis of proteins, (6) protease-related proteins concerning the protein breakdown, (7) proteins participating in replicating genome, (8) transcription factor participating in gene transcription, (9) nuclear protein participating in splicing mRNA, and so on. These proteins are not only important for elucidating the work of human cells but also useful in developing medicinals. Most of the small molecule medicinal so far known exhibit their pharmacological effects by combining with a particular protein existing in the cell and enhancing or inhibiting the action of the protein. Thus, possession of a set of human proteins becomes an effective instrument in screening these small molecule medicinals.

For getting human proteins there has so far been adopted a method of homogenizing human tissues or culture cells and purifying a single protein by combining various separating methods. Such proteins so far known as having a high content thereof and having been known to be active can be easily isolated and purified by conventional methods, but most of unknown proteins having a low content are difficult in isolation depending upon their quality. Further, most of human tissues are hardly available. Therefore, it is nearly impossible to provide every human protein in a conventional manner for isolation and purification.

On the other hand, the information on the structure of human proteins is described in human genome DNA, the primary structure of every human protein can be presumed if all of the information could be read. One of the objects of Human Genome Project consists herein. However, those obtained as a result of reading genome concern the information on DNA sequence only without proteins themselves. Within the cell, the genome information is at first transcribed into mRNA and the protein is synthesized by translating the sequence information of mRNA. Thus, if cDNA could be synthesized from the template mRNA, it is possible to synthesize the corresponding protein by using this cDNA. So, so-called EST project is going on, in which the partial base sequence of cDNA is determined by preparing cDNA from template mRNA isolated from various cells.

An essential requirement for cDNA in case of aiming at getting proteins is to involve all the translated region for the protein. That is, so-called full-length cDNA is required. However, the ratio of full-length cDNA is very low in cDNAs prepared by conventional methods, and it is also difficult to determine whether or not the cDNA is full-length. Thus, most of those known as EST are cDNA fragments containing only a part of the translated region for a protein.

On the other hand, the inventors of the present application established an original synthetic technology for full-length cDNA (Kato, S. et al., Gene 150:243-250, 1994). Then, human protein in full length can have been obtained by analyzing human full-length cDNA clone prepared by this technology. It is desirable to prepare the human protein bank by cloning all human full-length cDNA according to this technology.

Further, it has been elucidated as a result of search on human diseases seen so far that almost diseases are caused by having any disorder in the gene. For curing these diseases, the gene therapy of introducing normal gene in place of abnormal gene has been found promising. In this case, human full-length cDNA can be used as a gene source for the gene therapy.

In view of these circumstances above, the invention of the present application has been made, and its object is to provide a novel purified human protein, DNA fragments encoding the protein, expression vector for the DNA fragments, various cells transformed with the expression vector, and an antibody against the protein.

### DISCLOSURE OF INVENTION

The present application provides the following invention (i)-(ix).

(i) A purified human proteins having any one of the amino acid sequences of SEQ ID No: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158 or 160.

(ii) A DNA fragment encoding the protein of the invention (i).

(iii) A DNA fragment having the base sequence of the translated region in SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 30 109, 111, 113, 115, 117, 119, 121, 123, 125, 127, 129, 131, 133, 135, 137, 139, 141, 143, 145, 147, 149, 151, 153, 155, 157 or 159, which is human cDNA encoding the protein of the invention (i).

(iv) The DNA fragment of the invention (iii), which consists of any one of the base sequences of SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159.

(v) An expression vector, which is capable of expressing any one of the DNA fragments of inventions (ii) to (iv) by in vitro translation or within host cell.

(vi) The expression vector of the invention (v), which is capable of expressing a fused DNA fragment of the DNA fragment of any one of the invention (ii) to (iv) and the DNA fragment encoding a fluorescent protein.

(vii) A fluorescent protein-fused protein, which is an expression product of the expression vector of the invention (vi).

(viii) A cell transformed with the expression vector of the invention (v) or (vi), which is capable of producing the protein of the invention (i) or the fluorescent protein-fused protein of the invention (vii).

(ix) An antibody against the protein of the invention (i).

### BRIEF DESCRIPTION OF DRAWINGS

FIG. 1 is a drawing wherein the human protein encoded by clone HP02573 is compared with the amino acid sequence of bacteria GTP-binding protein CgpA.

FIG. 2 is a drawing wherein the human protein encoded by clone HP02612 is compared with the amino acid sequence of Mycobacteria 50S ribosomal protein L9.

FIG. 3 is a drawing wherein the human protein encoded by clone HP10117 is compared with the amino acid sequence of brucella ribosome recycling factor.

FIG. 4 is a drawing wherein the human protein encoded by clone HP10120 is compared with the amino acid sequence of nematoda hypothetical protein F45G2.10.

FIG. 5 is a drawing wherein the human protein encoded by clone HP10421 is compared with the amino acid sequence of nematoda hypothetical protein B0261.4.

FIG. 6 is a drawing wherein the human protein encoded by clone HP10582 is compared with the amino acid sequence of nematoda hypothetical protein 108.7 kDa.

FIG. 7 is a drawing wherein the human protein encoded by clone HP10149 is compared with the amino acid sequence of nematoda hypothetical protein W02A11.2.

FIG. 8 is a drawing wherein the human protein encoded by clone HP10160 is compared with the amino acid sequence of nematoda hypothetical protein ZK1248.15.

FIG. 9 is a drawing wherein the human protein encoded by clone HP10173 is compared with the amino acid sequence of nematoda hypothetical protein C04H5.1.

FIG. 10 is a drawing wherein the human protein encoded by clone HP02644 is compared with the amino acid sequence of nematoda RNA helicase-like protein.

FIG. 11 is a drawing wherein the human protein encoded by clone HP03233 is compared with the amino acid sequence of fission yeast putative ubiquinone biosynthesis methyltransferase.

FIG. 12 is a drawing wherein the human protein encoded by clone HP10437 is compared with the amino acid sequence of human pp21 homologue.

FIG. 13 is a drawing wherein the human protein encoded by clone HP10525 is compared with the amino acid sequence of fission yeast hypothetical protein SPAC8C9.11.

FIG. 14 is a drawing wherein the human protein encoded by clone HP10543 is compared with the amino acid sequence of mouse leucine-rich domain interacting protein 1.

FIG. 15 is a drawing wherein the human protein encoded by clone HP03090 is compared with the amino acid sequence of nematoda hypothetical protein 32.0 kDa.

FIG. 16 is a drawing wherein the human protein encoded by clone HP03145 is compared with the amino acid sequence of fission yeast mitochondrial p-hydroxybenzoate polyprenyltransferase-like protein.

FIG. 17 is a drawing wherein the human protein encoded by clone HP03185 is compared with the amino acid sequence of human histone macroH2A1.2.

FIG. 18 is a drawing wherein the human protein encoded by clone HP03324 is compared with the amino acid sequence of bacterial ribosomal protein L2.

FIG. 19 is a drawing wherein the human protein encoded by clone HP10648 is compared with the amino acid sequence of nematoda hypothetical protein Y40B1B.7.

FIG. 20 is a drawing wherein the human protein encoded by clone HP10162 is compared with the amino acid sequence of rat hypothetical protein.

FIG. 21 is a drawing wherein the human protein encoded by clone HP10334 is compared with the amino acid sequence of human SH3 domain binding glutamic acid-rich-like protein.

FIG. 22 is a drawing wherein the human protein encoded by clone HP10532 is compared with the amino acid sequence of human apoptosis associated protein Bbk.

FIG. 23 is a drawing wherein the human protein encoded by clone HP10559 is compared with the amino acid sequence of human hypothetical protein KIAA0276.

FIG. 24 is a drawing wherein the human protein encoded by clone HP10562 is compared with the amino acid sequence of human basic leucine-zipper protein LZIP.

FIG. 25 is a drawing wherein the human protein encoded by clone HP10456 is compared with the amino acid sequence of nematoda BC-2 like protein.

FIG. 26 is a drawing wherein the human protein encoded by clone HP10498 is compared with the amino acid sequence of nematoda hypothetical protein C24D19.6.

FIG. 27 is a drawing wherein the human protein encoded by clone HP10505 is compared with the amino acid sequence of nematoda hypothetical protein F29B9.10.

FIG. 28 is a drawing wherein the human protein encoded by clone HP10515 is compared with the amino acid sequence of drosophila hypothetical protein 63B12.s.

FIG. 29 is a drawing wherein the human protein encoded by clone HP01124 is compared with the amino acid sequence of human acyl-CoA-binding protein.

FIG. 30 is a drawing wherein the human protein encoded by clone HP02241 is compared with the amino acid sequence of African clawed frog's ribosomal protein L24-like protein.

FIG. 31 is a drawing wherein the human protein encoded by clone HP10101 is compared with the amino acid sequence of nematoda hypothetical protein C32E8.5.

FIG. 32 is a drawing wherein the human protein encoded by clone HP10370 is compared with the amino acid sequence of drosophila hypothetical protein CG11534.

FIG. 33 is a drawing wherein the human protein encoded by clone HP10427 is compared with the amino acid sequence of nematoda hypothetical protein Y106G6H.8.

FIG. 34 is a drawing wherein the human protein encoded by clone HP10516 is compared with the amino acid sequence of drosophila hypothetical protein CG14130.

FIG. 35 is a drawing wherein the human protein encoded by clone HP10580 is compared with the amino acid sequence of drosophila hypothetical protein CG5469.

### BEST MODE FOR CARRYING OUT THE INVENTION

The protein of the invention (i) can be obtained by a method of isolating it from human organs, cell line or the like, a method of preparing the peptide by chemical synthesis based on the amino acid sequence provided by the present application, a method of producing it by recombinant DNA techniques using DNA fragments of the inventions (ii)-(iv) above, and so on. Above all, the method by the recombinant DNA techniques is preferred. For example, the protein can be expressed in vitro by preparing RNA from the vector having the DNA fragment (cDNA) of the invention (iii) or (iv) above through in vitro transcription and performing in vitro translation using it as a template. Further, the protein encoded by the DNA fragment can be expressed in a large scale in prokaryotic cell such as Escherichia coli, Bacillus subtilis and the like or eukaryotic cell such as yeast, insect cell, mammalian cell, vegetable cell or the like, by subjecting the translated region to recombination in a conventional manner into an appropriate expression vector.

In case of producing the protein of the invention (i) by expressing DNA fragment through the in vitro translation, for example, the protein of the invention (i) above can be produced by subjecting the translated region of DNA fragment in the invention (iii) or (iv) above to recombination into the vector having RNA polymerase promotor and mixing it with the in vitro translation system such as rabbit reticulocyte lysate, wheat germ extract, or the like containing RNA polymerase corresponding to the promoter. The RNA polymerase promoter illustratively includes T7, T3, SP6 or the like. Examples of the vector containing these RNA polymerase promoters are pKA1, pCDM8, pT3/T7 18, pT7/3 19, pbluescipt II or the like.

In case of producing the protein of the invention (i) by expressing DNA fragment with microorganism such as E. coli, the protein desired can be produced in a large scale within a microorganism, for example, by preparing the recombinant expression vector wherein an expression vector having such an origin replicable in the microorganism, promoter, ribosome binding site, DNA cloning sites, terminator or the like is integrated with the translated region in the DNA fragment of the invention (iii) or (iv) above, transforming the host cell with this expression vector and cultivating the resultant transformant to give the protein encoded by this DNA fragment. In this case, the protein fragment containing an optional region can be obtained by expressing while adding the initiation codon and termination codon around the optional translated region. Otherwise, the protein can be also expressed as a fusion protein with other protein. Only the protein fragment encoded by this cDNA can be obtained by cutting this fusion protein with an appropriate protease. Examples of the expression vector for E. coli are pUC system, pBluescript II, pET expression system, pGEX expression system, and so on.

In case of producing the protein of the invention (i) by expressing DNA fragment with an eukaryotic cell, for example, the protein of the invention (i) above can be produced within the eukaryotic cell by recombining the translated region of DNA fragment in the invention (iii) or (iv) above within an expression vector for eukaryotic cell having promoter, splicing site, poly (A) addition site or the like and introducing it within the eukaryotic cell. Examples of the expression vector are pKA1, pCDM8, pSVK3, pMSG, pSVL, pBK-CMV, pBK-RSV, EBV vector, pRS, pYES2, and so on. Further, fusion protein added by various tags such as His tag, FLAG tag, GFP or the like can be also expressed by using pIND/V5-His, pFLAG-CMV-2, pEGFP-N1, pEGFP-C1 or the like as an expression vector. As the eukaryotic cells, mammalian cultured cells such as monkey kidney cells COS7 and Chinese hamster ovary cells CHO, budding yeasts, fission yeasts, silkworm cells and Xenopus oocytes are generally used, but insofar as the protein of the invention (1) can be expressed, any eukaryotic cells can be used. An expression vector can be induced into an eukaryotic cell in a conventional manner such as electroporation method, calcium phosphate method, liposome method, DEAE dextran method, or the like.

For isolating and purifying the protein of the invention (1) from a culture after expression of the desired protein in the prokaryotic or eukaryotic cells, separation techniques known in the art can be used in combination. Such techniques include e.g. treatment with a denaturant such as urea or a surfactant, sonication, enzymatic digestion, salting-out or solvent precipitation, dialysis, centrifugation, ultrafiltration, gel filtration, SDS-PAGE, isoelectric focusing, ion-exchange chromatography, hydrophobic chromatography, affinity chromatography and reverse phase chromatography.

The protein of the invention (i) includes also the peptide fragment (not less than 5 amino acid residues) consisting of any partial amino acid sequence in the SEQ ID No: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158 or 160. These peptide fragments can be used as an antigen for preparing an antibody. Further, most of the proteins of the invention (i) undergo various modification within the cell, after subjecting to translation. Thus, these modified proteins are also included in the scope of the protein of the invention (i). Examples of such modification after translation are elimination of N-terminal methionine, N-terminal acetylation, addition of sugar chain, limited proteolysis due to intracellular protease, myristoylation, isoprenylation, phophorylation, and so on.

The expression vector of the invention (v) is a vector that can translate in vitro the protein of the invention (i), as described above, or can express it within a host cell. Further, the expression vector of the invention (vi) is a vector that can express DNA fragment (the invention (ii), (iii) or (iv)) encoding the protein of the invention (i) and fused DNA fragment encoding the fluorescent protein. The fluorescent protein illustratively includes green fluorescent protein (GFP, EGFP), yellow fluorescent protein (EYFP), blue fluorescent protein (ECFP), red fluorescent protein (DsRed, the above-tradenames, Clontech Co.), green fluorescent protein coming from Renilla (hrGFP, Stratagene Co.) or the like. The position to fuse the fluorescent protein is either N-terminal or C-terminal of the protein. The expression vector of the invention (vi), being able to express a fusion protein of the protein of the invention (i) and the fluorescent protein (invention (vii)), is useful, for example, as a library for detecting the protein-protein interaction by using an intracellular localization site marker and 2-hybrid localization method.

DNA fragments (ii)-(iv) includes all DNA encoding the protein of the above (i). This DNA fragment can be obtained by using a chemical synthetic method, a cDNA cloning method, a method of screening human genome library, and so on.

DNA fragments (cDNA) in the invention (iii) or (iv) can be cloned, for example, cDNA library derived from human cell. The cDNA is prepared by using poly (A)⁺RNA extracted from human cell. Human cell may include those delivered from human body by means of operation or the like or those culture cells. The cDNA may be prepared by any synthetic methods such as Okayama-Berg method (Okayama, H. and Berg, P., Mol. Gell. Biol. 2: 161-170, 1982), Gubler-Hoffman method (Gubler, U. and Hoffman, J., Gene 25: 263-269, 1983) or the like, and preferably by Capping method (Kato, S. et al., Gene 150: 243-250, 1994), as shown in Example, for getting the full-length clone effectively. Further, commercially available human cDNA library can be also used. For cloning the objective cDNA from cDNA library, an oligonucleotide may be prepared on the basis of an optional part of base sequence from cDNA (SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159) of the invention (iii) or (iv) provided by the present invention, and then the screening due to the colony- or plaque-hybridization method may be performed by using it as a probe. Further, cDNA fragment of the invention (iii) or (iv) can be prepared also by preparing oligonucleotides to hybridize at each ends of the objective cDNA fragment and preparing it from mRNA isolated from human cell while using the oligonucleotide as a primer according to RT-PCR method.

The DNA fragment of the invention (iii) is cDNA having the base sequence of the translated region (Open Reading Frame: ORF) in SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159, and the DNA fragment of the invention (iv) is cDNA composed of any one of the base sequence of SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159. Table 1 collectively shows the respective sequence number, clone No. (HP No.), cell from which cDNA clone was obtained, number of all bases of cDNA, and number of amino acid residues in the protein encoded, respectively.

TABLE 1

| | | | | Number of |
|---|---|---|---|---|
| SEQ | | | Number of | Amino Acid |
| ID No:. | HP No. | Cell | Bases | Residue |
| 1, 2 | HP02573 | Stomach Carcinoma | 1323 | 284 |
| 3, 4 | HP02612 | Saos-2 | 1120 | 233 |
| 5, 6 | HP10021 | HT-1080 | 528 | 64 |
| 7, 8 | HP10117 | U937 | 1306 | 262 |
| 9, 10 | HP10120 | HT-1080 | 893 | 102 |
| 11, 12 | HP10321 | KB | 597 | 158 |
| 13, 14 | HP10416 | Stomach Carcinoma | 760 | 199 |
| 15, 16 | HP10421 | Stomach Carcinoma | 806 | 250 |
| 17, 18 | HP10582 | HT-1080 | 3907 | 614 |
| 19, 20 | HP10098 | U937 | 901 | 199 |
| 21, 22 | HP10106 | U937 | 1274 | 326 |
| 23, 24 | HP10111 | U937 | 1000 | 50 |
| 25, 26 | HP10149 | U937 | 1087 | 176 |
| 27, 28 | HP10151 | U-2 0S | 703 | 51 |
| 29, 30 | HP10160 | U937 | 921 | 190 |
| 31, 32 | HP10173 | HT-1080 | 584 | 125 |
| 33, 34 | HP10200 | HT-1080 | 875 | 176 |
| 35, 36 | HP10327 | KB | 470 | 52 |
| 37, 38 | HP02644 | HT-1080 | 2920 | 859 |
| 39, 40 | HP03233 | HT-1080 | 1502 | 327 |
| 41, 42 | HP10384 | KB | 737 | 86 |
| 43, 44 | HP10431 | Liver | 903 | 178 |
| 45, 46 | HP10437 | Stomach Carcinoma | 1170 | 117 |
| 47, 48 | HP10525 | Stomach Carcinoma | 404 | 86 |
| 49, 50 | HP10543 | HT-1080 | 752 | 179 |
| 51, 52 | HP10565 | Stomach Carcinoma | 1222 | 189 |
| 53, 54 | HP10570 | HT-1080 | 1209 | 117 |
| 55, 56 | HP03090 | KB | 1763 | 298 |
| 57, 58 | HP03115 | KB | 1913 | 358 |
| 59, 60 | HP03145 | KB | 1520 | 371 |
| 61, 62 | HP03185 | HT-1080 | 1731 | 372 |
| 63, 64 | HP03324 | U937 | 910 | 225 |
| 65, 66 | HP10052 | HT-1080 | 784 | 114 |
| 67, 68 | HP10626 | KB | 984 | 140 |
| 69, 70 | HP10633 | HT-1080 | 864 | 85 |
| 71, 72 | HP10637 | HT-1080 | 2617 | 579 |
| 73, 74 | HP10648 | KB | 1810 | 360 |
| 75, 76 | HP10211 | Saos-2 | 1620 | 126 |
| 77, 78 | HP10332 | Stomach Carcinoma | 1349 | 285 |
| 79, 80 | HP10641 | KB | 1355 | 329 |
| 81, 82 | HP10650 | KB | 1543 | 233 |
| 83, 84 | HP10654 | KB | 1436 | 183 |
| 85, 86 | HP10657 | U937 | 1357 | 380 |
| 87, 88 | HP10659 | U937 | 1399 | 260 |
| 89, 90 | HP10681 | HT-1080 | 1119 | 274 |
| 91, 92 | HP10077 | Stomach Carcinoma | 540 | 101 |
| 93, 94 | HP10162 | Saos-2 | 1059 | 278 |
| 95, 96 | HP10334 | HT-1080 | 782 | 93 |
| 97, 98 | HP10400 | Stomach Carcinoma | 417 | 57 |
| 99, 100 | HP10410 | Stomach Carcinoma | 697 | 115 |
| 101, 102 | HP10417 | Stomach Carcinoma | 1504 | 110 |
| 103, 104 | HP10482 | HT-1080 | 1046 | 133 |
| 105, 106 | HP10499 | Stomach Carcinoma | 341 | 68 |
| 107, 108 | HP10522 | Stomach Carcinoma | 1684 | 332 |
| 109, 110 | HP10532 | Stomach Carcinoma | 727 | 159 |
| 111, 112 | HP10552 | Saos-2 | 1354 | 245 |
| 113, 114 | HP10553 | HT-1080 | 653 | 110 |
| 115, 116 | HP10558 | Saos-2 | 643 | 123 |
| 117, 118 | HP10559 | Saoa-2 | 1293 | 237 |
| 119, 120 | HP10560 | Saos-2 | 916 | 107 |
| 121, 122 | HP10561 | Stomach Carcinoma | 1002 | 226 |
| 123, 124 | HP10562 | Saos-2 | 1753 | 395 |
| 125, 126 | HP10564 | Saos-2 | 668 | 22 |
| 127, 128 | HP10569 | KB | 279 | 70 |
| 129, 130 | HP10601 | HT-1080 | 3367 | 695 |
| 131, 132 | HP10456 | U-2 0S | 1290 | 199 |
| 133, 134 | HP10498 | Saos-2 | 564 | 118 |
| 135, 136 | HP10503 | Saos-2 | 904 | 114 |
| 137, 138 | HP10505 | Saos-2 | 472 | 87 |
| 139, 140 | HP10511 | Stomach Carcinoma | 180 | 39 |
| 141, 142 | HP10515 | Liver | 473 | 102 |
| 143, 144 | HP01124 | Liver | 1664 | 341 |
| 145, 146 | HP02241 | Stomach Carcinoma | 835 | 216 |
| 147, 148 | HP10101 | HT-1080 | 2465 | 396 |
| 149, 150 | HP10370 | KB | 3600 | 451 |
| 151, 152 | HP10427 | Stomach Carcinoma | 442 | 113 |
| 153, 154 | HP10438 | Stomach Carcinoma | 726 | 222 |
| 155, 156 | HP10502 | HT-1080 | 1120 | 278 |
| 157, 158 | HP10516 | Stomach Carcinoma | 747 | 221 |
| 159, 160 | HP10580 | Stomach Carcinoma | 1441 | 441 |

Further, the same clone as cDNA of the invention (iii) and (iv) can be easily prepared by screening the cDNA library prepared from human cell line as shown in Table 1 or human tissue while using the oligonucleotide probe prepared on the basis of any one of the base sequence, SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159.

Furthermore, polymorphisms are generally often observed in human genes. Thus, in SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159, the cDNA in which one or more nucleotides are added, deleted and/or substituted by other nucleotides is included in the scope of the invention.

Similarly, the protein in which one or more amino acids are added, deleted and/or substituted by other amino acids brought about by these modifications are included in the scope of the present invention, as far as it possess the activity of the protein having the amino acid sequence in SEQ ID No: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158 or 160.

The DNA fragment (not less than 10 bp) having any partial base sequence in SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159 is also included in the DNA fragment of the invention (iii) or (iv). Further, the DNA fragment composed of sense strand and anti-sense strand is also included in this scope. These DNA fragments can be used as a probe for the gene diagnosis.

The antibody of the invention (vii) can be obtained from serum after immunizing an animal by using the protein of the invention (i) as an antigen. As an antigen, the peptide prepared chemically on the basis of amino acid sequence in SEQ ID No: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158 or 160, as well as the protein expressed in eukaryotic cell or prokaryotic cell can be used as an antigen. Otherwise, the antigen can be prepared by introducing the expression vector for eukaryotic cell into animals' muscle or skin by injection or gene gun and collecting the serum (e.g. the method described in Japanese Patent Application Provisional Publication No. 7-313187). Examples of the animal are mouse, rat, rabbit, goat, chick, and so on. The monoclonal antibody corresponding to the protein of the invention (i) by preparing a hybridoma while fusing the myeloma with B cell collected from the spleen of the immunized animal.

### EXAMPLES

The present invention will be described in more detail by reference to the Examples, which however are not intended to limit the scope of the present invention. Basic procedures for DNA recombination and enzymatic reaction were in accordance with those described in a literature (Molecular Cloning, A Laboratory Manual, Cold Spring Harbor Laboratory, 1989). Unless otherwise specified, the restriction enzymes and various modifying enzymes used were products of Takara Shuzo Co., Ltd. The buffer composition in each enzymatic reaction, as well as reaction conditions, was followed instructions attached to the kits. Synthesis of cDNA was conducted according to a literature (Kato, S. et al., Gene, 150, 243-250, 1994).

### Example 1

### cDNA Cloning

As cDNA library, human full-length cDNA library (WO 97/33993, WO98/11217, and WO98/21328 Gazette) was used. From the individual library full-length cDNA clones were selected and the total base sequence was determined. The details of the obtained clones 1 to 80 will be described below.

1: HP02573

As the result of determining the total base sequence of cDNA insert of clone HP02573 obtained from human stomach carcinoma cDNA library, it was found that it had a structure of 26 bp 5′ untranslated region, 855 bp ORF and 442 bp 3′ untranslated region (SEQ ID No: 1). The ORF encodes the protein consisting of 284 amino acid residues (SEQ ID No: 2), and as a result of in vitro translation, the translated product of almost the same 31 kDa as molecular weight 32,126 anticipated from the ORF was produced (Example 2). The fusion protein of this protein and GFP was observed to be expressed in the whole cell (Example 4).

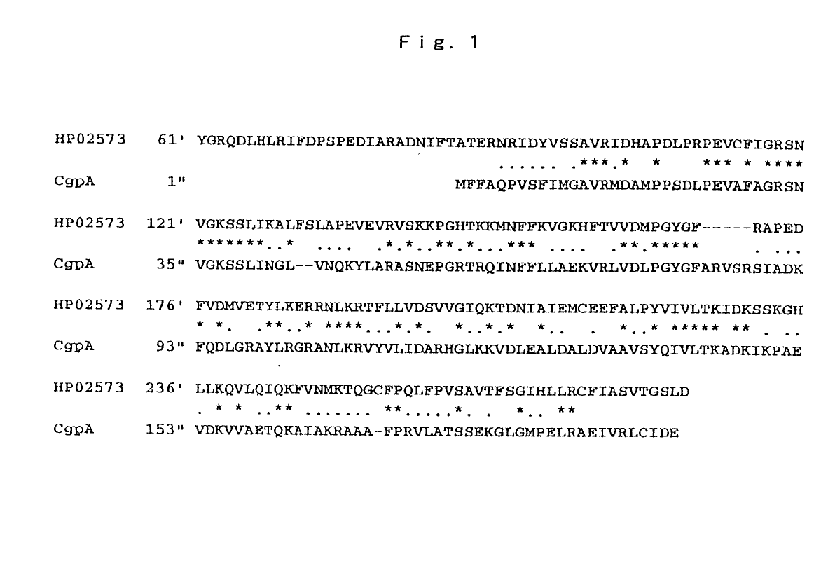

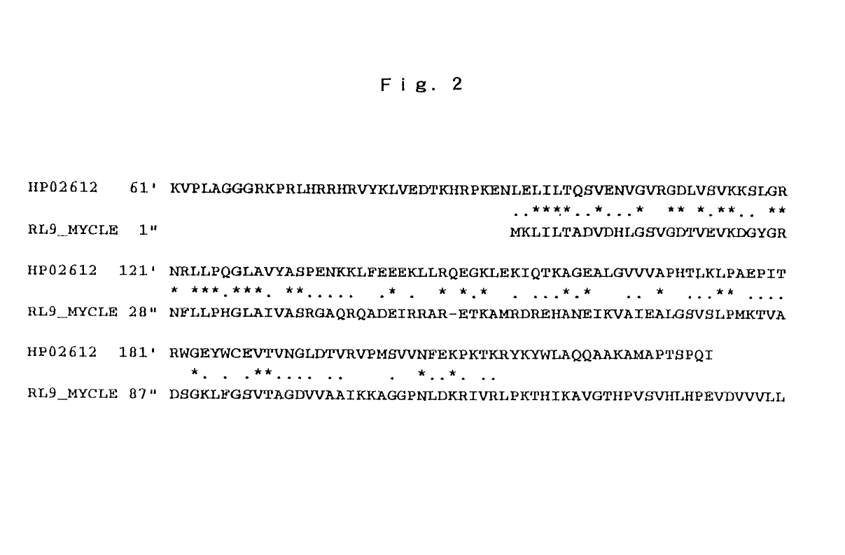

As a result of searching the protein database by using the amino acid sequence of this protein, there was found a similarity to bacteria GTP-binding protein CgpA (Accession No. AAC69623). FIG. 1 shows the comparison of amino acid sequence between the human protein encoded by the clone 1 and the bacteria GTP-binding protein CgpA. In this figure, - means a gap, * means the same amino acid residue as the protein of this invention, and . means the amino acid residue similar to the protein of this invention, respectively. Over the whole region except for the N-terminal region, they had a homology of 37.2%.

Further, as a result of reference to GenBank on the basis of the base sequence of clone 1 cDNA, those having a homology of not less than 90% (e.g. Accession No. AA429983) were found to have been registered in EST, but as it is of the partial sequence, it cannot be decided whether or not the same protein as that encoded by clone 1 is encoded.

GTP-binding protein plays an important role in route of the intracellular signal transduction.

2: HP02612

As the result of determining the total base sequence of cDNA insert of clone HP02612 obtained from human osteosarcoma cell line Saos-2 cDNA library, it was found that it had a structure of 17 bp 5′ untranslated region, 702 bp ORF and 401 bp 3′ untranslated region was found (SEQ ID No: 3). The ORF encoded the protein consisting of 233 amino acid residues (SEQ ID No: 4), and as a result of in vitro translation, the translated product of 29 kDa slightly larger than molecular weight 26,038 anticipated from the ORF was produced (Example 2). The fusion protein of this protein and GFP was found to be localized in the mitochondria (Example 4).

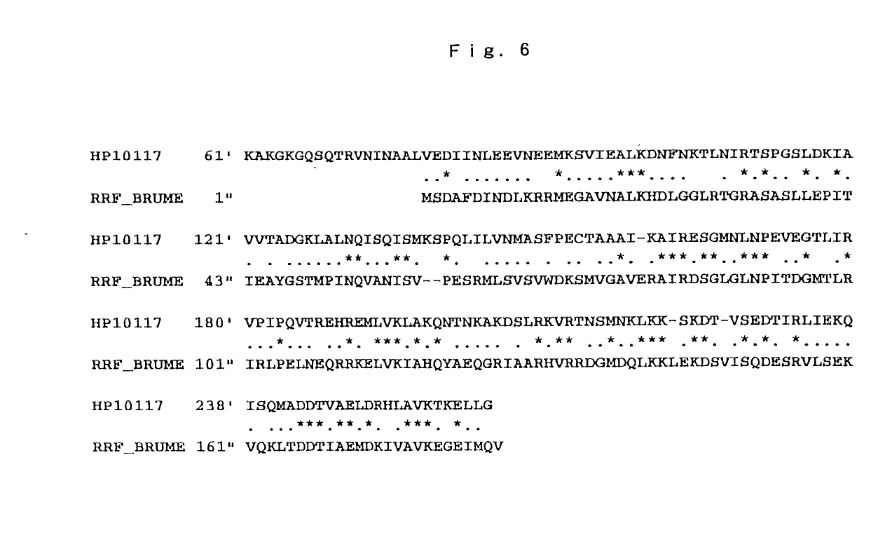

As a result of searching the protein database by using the amino acid sequence of this protein, there was found a similarity to Mycobacterium 50S ribosomal protein L9 (Accession No. P46385). FIG. 2 shows the comparison of amino acid sequence between the human protein encoded by clone 2 and mycobacteria 50S ribosomal protein L9. In this figure, - means a gap, * means the same amino acid residue as the protein of this invention, and . means an amino acid residue similar to the protein of this invention, respectively. Over the whole region except for the N-terminal region, they had a homology of 30.3%. In reconsidering the localization to mitochondria, it is considered that this protein is a mitochondria ribosomal protein, and the N-terminal region is a signal sequence for mitochondrial localization.

Further, as a result of reference to GenBank on the basis of the base sequence of clone 2 cDNA, those having a homology of not less than 90% (e.g. Accession No. H79400) were found to have been registered in EST, but as it is of the partial sequence, it cannot be decided whether or not the same protein as that encoded by clone 2 is encoded.

The mitochondria ribosomal protein is one of the proteins constituting the mitochondria ribosome and participates in the translation system within the mitochondria.

3: HP10021

As the result of determining the total base sequence of cDNA insert of clone HP10021 obtained from human fibrosarcoma cell line HT-1080 cDNA library, it was found that it had a structure of 89 bp 5′ untranslated region, 195 bp ORF and 244 bp 3′ untranslated region (SEQ ID No: 5). The ORF encoded the protein consisting of 64 amino acid residues (SEQ ID No: 6). The fusion protein of this protein and GFP was observed to be expressed in the whole cell (Example 4).

Further, as a result of reference to GenBank on the basis of the base sequence of clone 3 cDNA, those having a homology of not less than 90% (e.g. Accession No. AA156954) were found to have been registered in EST, but as it is of the partial sequence, it cannot be decided whether or not the same protein as that encoded by clone 3 is encoded.

4: HP10117

As the result of determining the total base sequence of cDNA insert of clone HP10117 obtained from human lymphoma cell line U937 cDNA library, it was found that it had a structure of 52 bp 5′ untranslated region, 789 bp ORF and 465 bp 3′ untranslated region (SEQ ID No: 7). The ORF encoded the protein consisting of 262 amino acid residues (SEQ ID No: 8), and as a result of in vitro translation, a translated product of 30 kDa almost same as molecular weight 29,259 anticipated from the ORF was produced (Example 2). The fusion protein of this protein and GFP was found to be localized in the mitochondria (Example 4).

As a result of searching the protein database by using the amino acid sequence of this protein, there was found a similarity to brucella ribosome recycling factor (Accession No. P94340). FIG. 3 shows the comparison of the amino acid sequence between the human protein encoded by clone 4 and the brucella ribosomal recycling factor. In this figure, - means a gap, * means the same amino acid residue as the protein of this invention, and . means an amino acid residue similar to the protein of this invention, respectively. Over the whole region except for the N-terminal region, they had a homology of 29.0%. In reconsidering the localization to mitochondria, it is considered that this protein is a mitochondria ribosomal recycling factor, and the N-terminal region is a signal sequence for mitochondrial localization.

Further, as a result of referring to GenBank on the basis of the base sequence of clone 4 cDNA, those having a homology of not less than 90% (e.g. Accession No. H67316) were found to have been registered in EST, but as it is of the partial sequence, it cannot be decided whether or not the same protein as that encoded by clone 4 is encoded.

The ribosomal recycling factor is a factor needed for removing mRNA from ribosome at the time of finishing the protein synthesis, and works for enhancing the translational efficiency on the ribosome.

5: HP10120

As the result of determining the total base sequence of cDNA insert of clone HP10120 obtained from human fibrosarcoma cell line HT-1080 cDNA library, it was found that it had a structure of 95 bp 5′ untranslated region, 309 bp ORF and 489 bp 3′ untranslated region (SEQ ID No: 9). The ORF encoded the protein consisting of 102 amino acid residues (SEQ ID No: 10), and as a result of in vitro translation, a translated product of 14 kDa slightly larger than molecular weight 11,634 anticipated from the ORF was produced (Example 2). The fusion protein of this protein and GFP was observed to be expresses in the whole cell (Example 4).

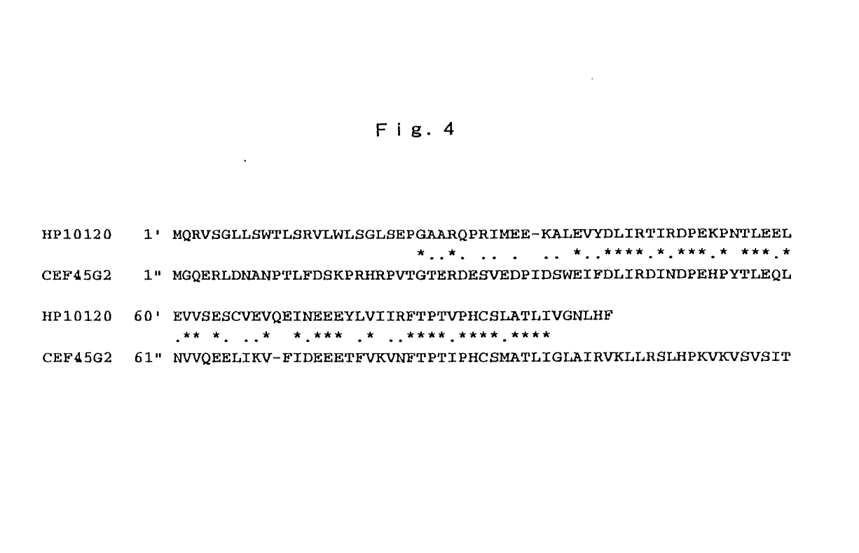

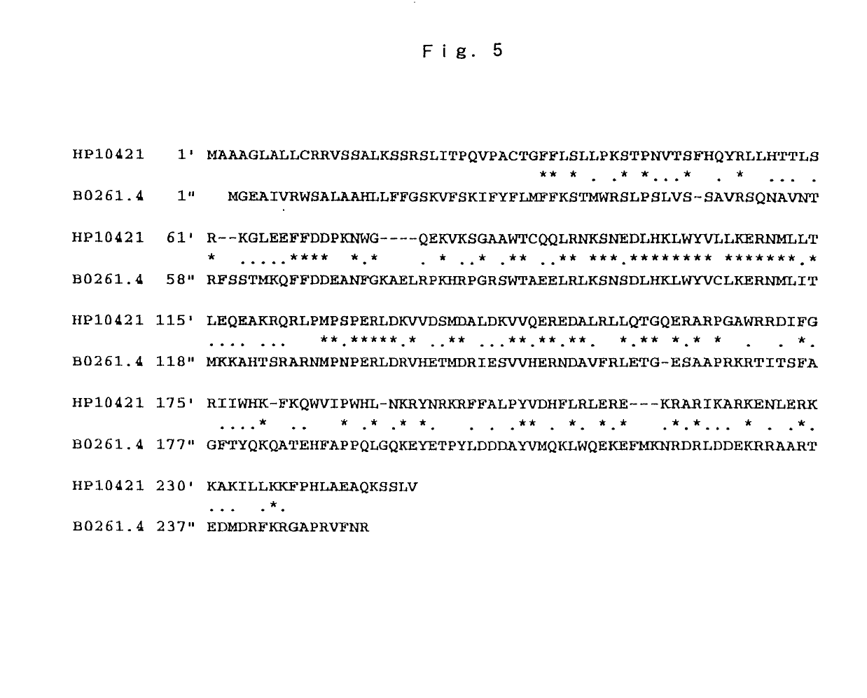

As a result of searching the protein database by using the amino acid sequence of this protein, there was found a similarity to nematode hypothetical protein F45G2.10 (Accession No. CBA07619). FIG. 4 shows the comparison of the amino acid sequence between the human protein encoded by clone 5 and the nematode hypothetical protein F45G2.10. In this figure, - means a gap, * means the same amino acid residue as the protein of this invention, and . means the same amino acid residue as the protein of this invention, respectively. Over the C-terminal 73 amino acid residues, they had a homology of 50.7%.

Further, as a result of referring to GenBank on the basis of the base sequence of clone 5 cDNA, those having a homology of not less than 90% (e.g. Accession No. N44558) were found to have been registered in EST, but as it is of the partial sequence, it cannot be decided whether or not the same protein as that encoded by clone 5 is encoded.

6: HP10321

As the result of determining the total base sequence of cDNA insert of clone HP10321 obtained from human epidermal carcinoma cell line KB cDNA library, it was found that it had a structure of 20 bp 5′ untranslated region, 477 bp ORF and 100 bp 3′ untranslated region (SEQ ID No: 11). The ORF encoded the protein consisting of 158 amino acid residues (SEQ ID No:. 12), and as a result of in vitro translation, the translated product of 19 kDa slightly larger than molecular weight 16,215 anticipated from the ORF was produced (Example 2). The fusion protein of this protein and GFP was observed to be expressed in the whole cell (Example 4).

As a result of searching GenBank on the basis of base sequence of cDNA of clone 6, those having a homologue of not less than 90% (Accession No. AA010288) were found to have been registered in EST, but as it is of the partial sequence, it cannot be decided whether or not the same protein as that encoded by clone 6 is encoded.

7: HP10416

As the result of determining all the base sequence of cDNA insert of clone HP10416 obtained from human stomach carcinoma cDNA library, it was found that it had a structure of 96 bp 5′ untranslated region, 600 bp ORF and 64 bp 3′ untranslated region (SEQ ID No: 13). The ORF encoded the protein consisting of 199 amino acid residues (SEQ ID No: 14), and as a result of in vitro translation, a translated product of 23 kDa almost same as molecular weight 22,340 anticipated from the ORF was produced (Example 2). The fusion protein of this protein and GFP was found to be localized as particles in the nucleus or cytoplasm (Example 4).

Further, as a result of referring to GenBank on the basis of the base sequence of clone 7 cDNA, those having a homology of not less than 90% (e.g. Accession No. AA218581) were found to have been registered in EST, but as it is of the partial sequence, it cannot be decided whether or not the same protein as that encoded by clone 7 is encoded.

8: HP10421

As the result of determining the total base sequence of cDNA insert of clone HP10421 obtained from human stomach carcinoma cDNA library, it was found that it had a structure of 14 bp 5′ untranslated region, 753 bp ORF and 39 bp 3′ untranslated region (SEQ ID No: 15). The ORF encoded the protein consisting of 250 amino acid residues (SEQ ID No: 16), and as a result of in vitro translation, a translated product of almost the same 30 kDa as molecular weight 29,450 anticipated from the ORF was produced (Example 2). The fusion protein of this protein and GFP was found to be localized in the mitochondria (Example 4).

As the result of searching the protein database by using the amino acid sequence of this protein, there was found to have a similarity to nematoda hypothetical protein B0261.4 (Accession No.AAB52351). FIG. 5 shows a comparison of the amino acid sequence between the human protein encoded by clone 8 and the nematoda hypothetical protein B0261.4. In this figure, - means a gap, * means the same amino acid residue as the protein of this invention, and . means the amino acid residue similar to the protein of this invention, respectively. Over the whole region except for the N-terminal region they had a homology of 35.8%. Further, there was also found a similarity to mitochondria 60S ribosomal protein L4 in yeast. In view of localization in the mitochondria, it is considered that this protein is one of mitochondria ribosomal proteins, in which N-terminal region takes a signal sequence for mitochondrial localization.

Further, as a result of referring to GenBank on the basis of the base sequence of cDNA of clone 8, those having a homology of not less than 90% (e.g. Accession No. AA167086) were found to have been registered in EST, but as it is of the partial sequence, it cannot be decided whether or not the same protein as that encoded by clone 8 is encoded.

The mitochondria ribosomal protein is one of the proteins constituting a mitochondria ribosome and participates in the translational system within the mitochondria.

9: HP10582

As the result of determining the total base sequence of cDNA insert of clone HP10582 obtained from human fibrosarcoma cell line HT-1080 cDNA library, it was found that it had a structure of 131 bp 5′ untranslated region, 1845 bp ORF and 1931 bp 3′ untranslated region (SEQ ID No: 17). The ORF encoded the protein consisting of 614 amino acid residues (SEQ ID No: 18), and as a result of in vitro translation, a translated product of 70 kDa almost same as molecular weight 69,774 anticipated from the ORF was produced (Example 2). A reticular expression was found in the cytoplasm on the fusion protein of this protein and GFP (Example 4).

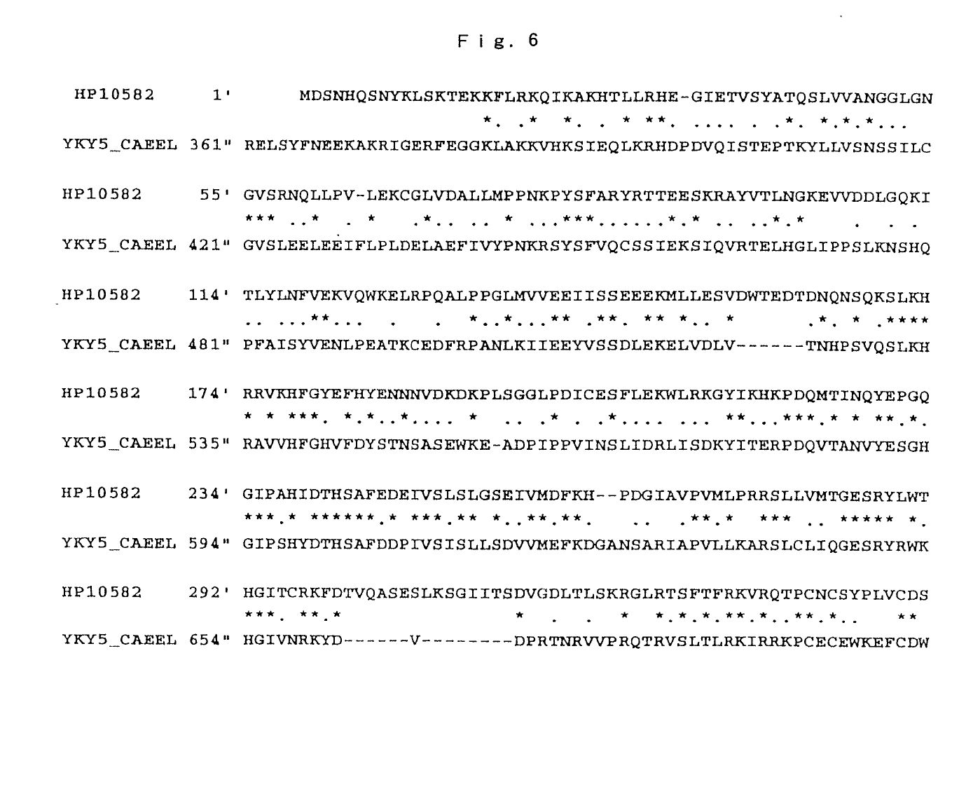

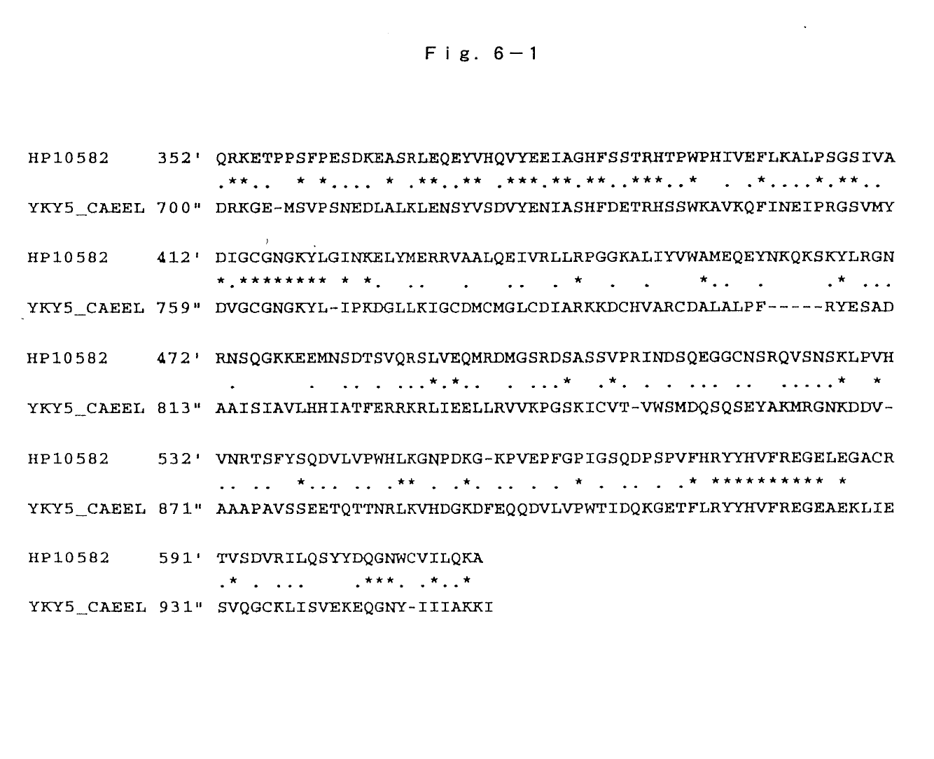

As a result of searching the protein database by using the amino acid sequence of this protein, there was found a similarity to nematoda hypothetical protein 108.7 kDa (Accession No. P49958). FIG. 6 shows a comparison of the amino acid sequence between the human protein encoded by clone 9 and the nematoda hypothetical protein 108.7 kDa. In this figure, - means a gap, * means the same amino acid residue as the protein of this invention, and . means an amino acid residue similar to the protein of this invention, respectively. Over the C-terminal region 602 amino acid residues, they had a homology of 30.2%.

Further, as a result of referring to GenBank on the basis of the base sequence of cDNA of clone 9, those having a homology of not less than 90% (e.g. Accession No. AA313350) were found to have been registered in EST, but as it is of the partial sequence, it cannot be decided whether or not the same protein as that encoded by clone 9 is encoded.

10: HP10098

As the result of determining the total base sequence of cDNA insert of clone HP10098 obtained from human lymphoma cell line U937 cDNA library, it was found that it had a structure of 35 bp 5′ untranslated region, 600 bp ORF and 266 bp 3′ untranslated region (SEQ ID No: 19). The ORF encoded the protein consisting of 199 amino acid residues (SEQ ID No: 20), and as a result of in vitro translation, a translated product of 24 kDa slightly larger than molecular weight 21,750 anticipated from the ORF was produced (Example 2). A particle expression was found in the cytoplasm on the fusion protein of this protein and GFP (Example 4).

Further, as a result of referring to GenBank on the basis of the base sequence of clone 10 cDNA, those having a homology of not less than 90% (e.g. Accession No. H40208) were found to have been registered in EST, but as it is of the partial sequence, it cannot be decided whether or not the same protein as that encoded by clone 10 is encoded. Moreover, a clone (Accession No. AX014145, WO 9954447-A) showing a homology of 99.7% was found to have been registered, but this clone encodes a protein different from clone 10, because the clone causes the frame shift due to shortage of G corresponding to 139th in clone 10.

11: HP10106

As the result of determining the total base sequence of cDNA insert of clone HP10106 obtained from human lymphoma cell line U937 cDNA library, it was found that it had a structure of 130 bp 5′ untranslated region, 981 bp ORF and 163 bp 3′ untranslated region (SEQ ID No: 21). The ORF encoded the protein consisting of 362 amino acid residues (SEQ ID No: 22), and as a result of in vitro translation, a translated product of 41 kDa slightly larger than molecular weight 36,684 anticipated from the ORF was produced (Example 2). The fusion protein of this protein and GFP was found to be expressed in the whole cell (Example 4).

Further, as a result of referring to GenBank on the basis of the base sequence of cDNA of clone 11, those having a homology of not less than 90% (e.g. Accession No. AA384225) were found to have been registered in EST, but as it is of the partial sequence, it cannot be decided whether or not the same protein as that encoded by clone 11 is encoded.

12: HP10111

As the result of determining the total base sequence of cDNA insert of clone HP10111 obtained from human lymphoma cell line U937 cDNA, it was found that it had a structure of 32 bp 5′ untranslated region, 153 bp ORF and 815 bp 3′ untranslated region (SEQ ID No: 23). The ORF encoded the protein consisting of 50 amino acid residues (SEQ ID No: 24), and as a result of in vitro translation, a translated product of 6 kDa almost same as molecular weight 5,547 anticipated from the ORF was produced (Example 2). The fusion protein of this protein and GFP was found to be expressed in reticular form in the whole cell (Example 4).

Further, as a result of referring to GenBank on the basis of the base sequence of cDNA of clone 12, those having a homology of not less than 90% (e.g. Accession No. AL110141) were found to have been registered in EST, but as it is of the partial sequence, it cannot be decided whether or not the same protein as that encoded by clone 12 is encoded.

13: HP10149

As the result of determining the total base sequence of cDNA insert of clone HP10149 obtained from human lymphoma cell line U937 cDNA library, it was found that it had a structure of 27 bp 5′ untranslated region, 531 bp ORF and 529 bp 3′ untranslated region (SEQ ID No: 25). The ORF encoded the protein consisting of 176 amino acid residues (SEQ ID No: 26), and as a result of in vitro translation, a translated product of 23 kDa slightly larger than molecular weight 20,734 anticipated from the ORF was produced (Example 2). The fusion protein of this protein and GFP was found to be expressed in the whole cell (Example 4).

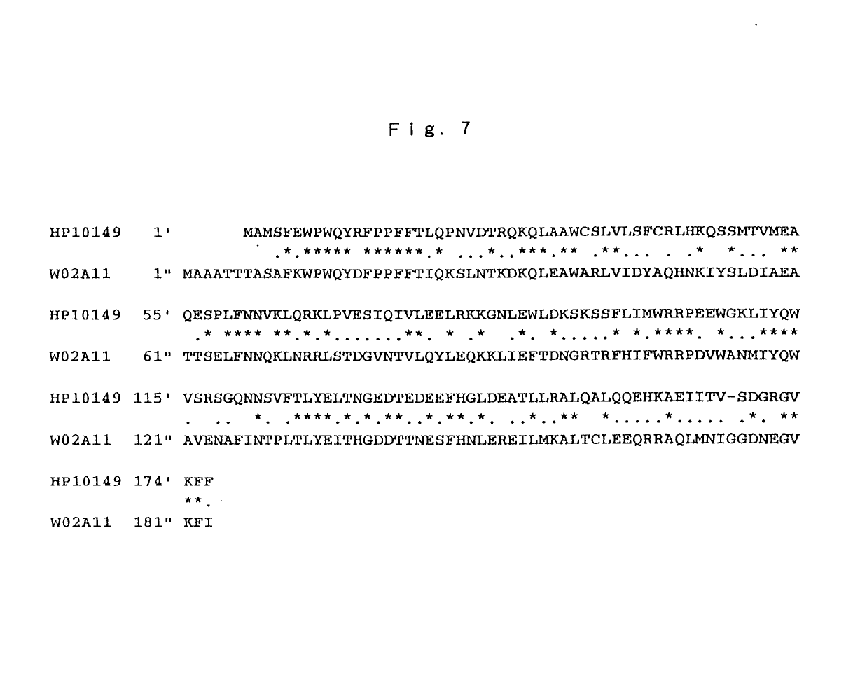

As a result of searching the protein database by using the amino acid sequence of this protein, there was found a similarity to nematoda hypothetical protein W02A11.2 (Accession No. CAB04889). FIG. 7 shows a comparison of the amino acid sequence between human protein encoded by clone 13 and nematoda hypothetical protein W02A11.2. In this figure, - means a gap, * means the same amino acid residue as the protein of this invention, and . means an amino acid residue similar to the protein of this invention, respectively. Over the whole region, they had a homology of 42.5%.

Further, as a result of referring to GenBank on the basis of the base sequence of cDNA of clone 13, those having a homology of not less than 90% (e.g. Accession No. T34989) were found to have been registered in EST, but as it is of the partial sequence, it cannot be decided whether or not the same protein as that encoded by clone 13 is encoded. Moreover, a clone (Accession No. AR070327, U.S. Pat. No. 5,892,010) having the same partial sequence was found to have been registered, but there is no decision whether or not it is encoded by the same protein as that encoded by clone 13.

14: HP10151

As the result of determining the total base sequence of cDNA insert of clone HP10151 obtained from human osteosarcoma cell line U-2 OS cDNA library, it was found that it had a structure of 66 bp 5′ untranslated region, 156 bp ORF and 481 bp 3′ untranslated region (SEQ ID No: 27). The ORF encoded the protein consisting of 51 amino acid residues (SEQ ID No: 28), and as a result of in vitro translation, a translated product of 6 kDa almost same as molecular weight 6,031 anticipated from the ORF was produced (Example 2). The fusion protein of this protein and GFP was found to be localized in the golgi body (Example 4).

Further, as a result of referring to GenBank on the basis of the base sequence of cDNA of clone 14, those having a homology of not less than 90% (e.g. Accession No. AA304503) were found to have been registered in EST, but as it is of the partial sequence, it cannot be decided whether or not the same protein as that encoded by clone 14 is encoded.

15: HP10160

As the result of determining the total base sequence of cDNA insert of clone HP10160 obtained from human lymphoma cell line U937 cDNA library, it was found that it had a structure of 203 bp 5′ untranslated region, 573 bp ORF and 145 bp 3′ untranslated region (SEQ ID No: 29). The ORF encoded the protein consisting of 190 amino acid residues (SEQ ID No: 30), and as a result of in vitro translation, a translated product of 25 kDa slightly larger than molecular weight 21,481 anticipated from the ORF was produced (Example 2). The fusion protein of this protein and GFP was found to be expressed in the whole cell (Example 4).

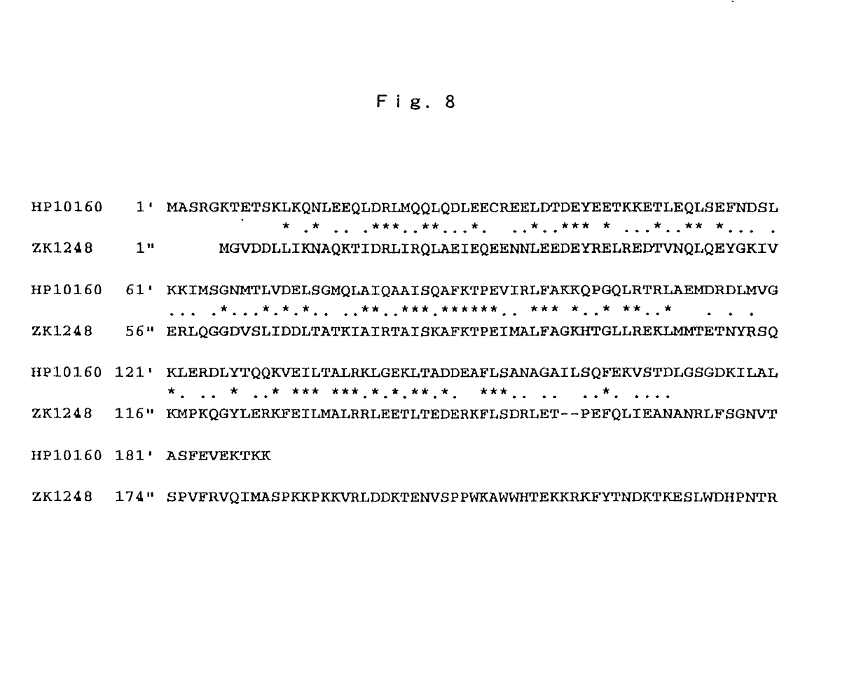

As a result of searching the protein database by using the amino acid sequence of this protein, there was found a similarity to nematoda hypothetical protein ZK1248.15 (Accession No. AAC71096). FIG. 8 shows a comparison of the amino acid sequence between the human protein encoded by clone 15 and the nematoda hypothetical protein ZK1248.15. In this figure, - means a gap, * means the same amino acid residue as the protein of this invention, and . means an amino acid residue similar to the protein of this invention, respectively. Over the N-terminal 159 amino acid residue, they had a homology of 36.5%.

Further, as a result of referring to GenBank on the basis of the base sequence of cDNA of clone 15, those having a homology of not less than 90% (e.g. Accession No. AA304503) were found to have been registered in EST, but as it is of the partial sequence, it cannot be decided whether or not the same protein as that encoded by clone 15 is encoded.

16: HP10173

As the result of determining the total base sequence of cDNA insert of clone HP10173 obtained from human fibrosarcoma cell line HT-1080 cDNA library, it was found that it had a structure of 40 bp 5′ untranslated region, 378 bp ORF and 166 bp 3′ untranslated region (SEQ ID No: 31). The ORF encoded the protein consisting of 125 amino acid residues (SEQ ID No: 32), and as a result of in vitro translation, a translated product of 15 kDa almost same as molecular weight 14,190 anticipated from the ORF was produced (Example 2). The fusion protein of this protein and GFP was found to be expressed in the whole cell (Example 4).

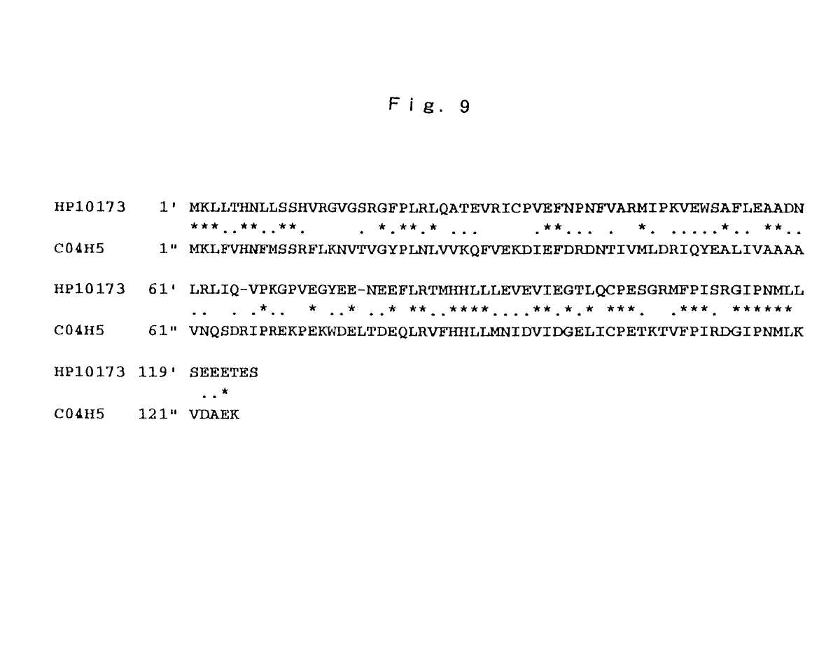

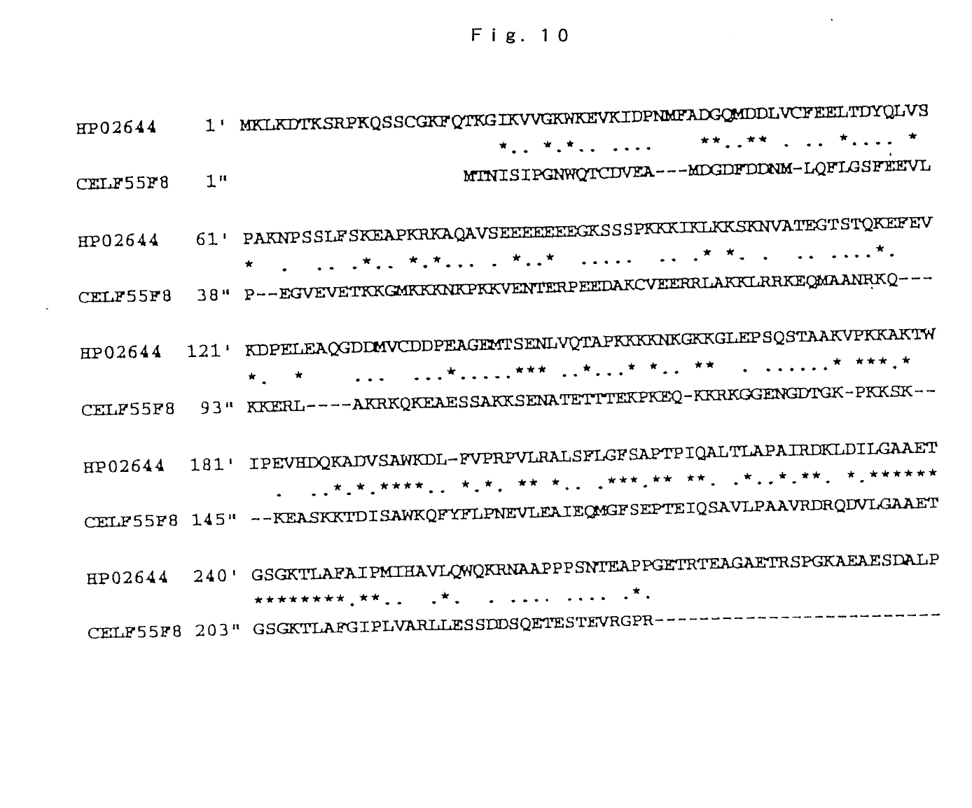

As a result of searching the protein database by using the amino acid sequence of this protein, there was found a similarity to nematoda hypothetical protein C04H5.1 (Accession No. CAB03840). FIG. 9 shows a comparison of the amino acid sequence between the human protein encoded by clone 16 and the nematoda hypothetical protein C04H5.1. In this figure, - means a gap, * means the same amino acid residue as the protein of this invention, and . means an amino acid residue similar to the protein of this invention, respectively. Over the whole region, they had a homology of 35.5%.

Further, as a result of referring to GenBank on the basis of the base sequence of cDNA of clone 16, those having a homology of not less than 90% (e.g. Accession No. AA937773) were found to have been registered in EST, but as it is of the partial sequence, it cannot be decided whether or not the same protein as that encoded by clone 16 is encoded. Moreover, a clone (Accession No. AX011631, WO 9955858-A) having the same partial sequence was found to have been registered, but this clone has ORF different from that of clone 16, its 5′-terminal being by 199 bp longer than that of clone 16.

17: HP10200

As the result of determining the total base sequence of cDNA insert of clone HP10200 obtained from human fibrosarcoma cell line HT-1080 cDNA library, it was found that it had a structure of 24 bp 5′ untranslated region, 531 bp ORF and 320 bp 3′ untranslated region (SEQ ID No: 33). The ORF encoded the protein consisting of 176 amino acid residues (SEQ ID No: 34), and as a result of in vitro translation, a translated product of 24 kDa slightly larger than molecular weight 18,408 anticipated from the ORF was produced (Example 2). The fusion protein of this protein and GFP was found to be expressed in the whole cell (Example 4).

Further, as a result of referring to GenBank on the basis of the base sequence of cDNA of clone 17, those having a homology of not less than 90% (e.g. Accession No. AA187416) were found to have been registered in EST, but as it is of the partial sequence, it cannot be decided whether or not the same protein as that encoded by clone 17 is encoded. Moreover, a clone (Accession No. AX015360, WO 9951727-A) having a homology of 95.6% was found to have been registered, but this clone encodes a protein different from that of clone 17, because a frame shift brings about due to shortage of C corresponding to 53rd of clone 17.

18: HP10327

As the result of determining the total base sequence of cDNA insert of clone HP10327 obtained from human epidermal carcinoma cell line KB cDNA library, it was found that it had a structure of 215 bp 5′ untranslated region, 159 bp ORF and 96 bp 3′ untranslated region (SEQ ID No: 35). The ORF encoded the protein consisting of 52 amino acid residues (SEQ ID No: 36), and as a result of in vitro translation, a translated product of 6 kDa almost same as molecular weight 5,636 anticipated from the ORF was produced (Example 2). The fusion protein of this protein and GFP was found to be expressed in the reticular form in the cytoplasm (Example 4).

Further, as a result of referring to GenBank on the basis of the base sequence of cDNA of clone 18, those having a homology of not less than 90% (e.g. Accession No. A1097092) were found to have been registered in EST, but as it is of the partial sequence, it cannot be decided whether or not the same protein as that encoded by clone 18 is encoded.

19: HP02644

As the result of determining the total base sequence of cDNA insert of clone HP02644 obtained from human fibrosarcoma cell line HT-1080 cDNA library, it was found that it had a structure of 72 bp 5′ untranslated region, 2580 bp ORF and 268 bp 3′ untranslated region (SEQ ID No: 37). The ORF encoded the protein consisting of 859 amino acid residues (SEQ ID No: 38), and as a result of in vitro translation, a translated product of 150 kDa larger than molecular weight 96,271 anticipated from the ORF was produced (Example 2). The fusion protein of this protein and GFP was found to be expressed in the nucleolus (Example 4).

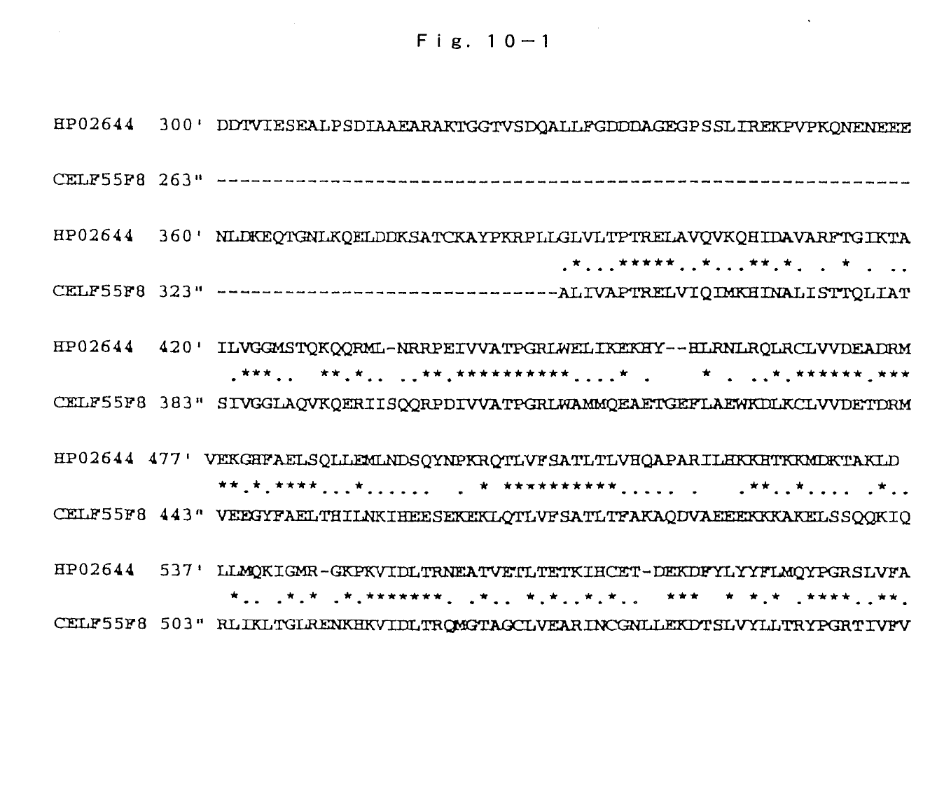

As a result of searching the protein database by using the amino acid sequence of this protein, there was found a similarity to nematoda RNA helicase-like protein CELF55F8 (Accession No. AAB37806). FIG. 10 shows a comparison of the amino acid sequence between the human protein encoded by clone 19 and the nematoda RNA helicase-like protein. In this figure, - means a gap, * means the same amino acid residue as the protein of this invention, and . means an amino acid residue similar to the protein of this invention, respectively. Over the whole region, they had a homology of 31.6%. The RNA helicase-like protein participates in many processes that RNA concerns, such as ribosome formation, transcription, splicing, RNA maturing, RNA transportation, RNA disassimilation, translation or the like.

Further, as a result of referring to GenBank on the basis of the base sequence of cDNA of clone 19, those having a homology of not less than 90% (e.g. Accession No. Z48570 or A74673) were found to have been registered in EST, but any is short than cDNA of clone 19. Also those having a homology of not less than 90% (e.g. Accession No. AA788907) were found to have been registered in EST, but as it is of the partial sequence, it cannot be decided whether or not the same protein as that encoded by clone 19 is encoded.

20: HP03233

As the result of determining the total base sequence of cDNA insert of clone HP03233 obtained from human fibrosarcoma cell line HT-1080 cDNA library, it was found that it had a structure of 14 bp 5′ untranslated region, 984 bp ORF and 504 bp 3′ untranslated region (SEQ ID No: 39). The ORF encoded the protein consisting of 327 amino acid residues (SEQ ID No: 40), and as a result of in vitro translation, a translated product of 37 kDa almost same as molecular weight 37,116 anticipated from the ORF was produced (Example 2). The fusion protein of this protein and GFP was found to be expressed in the Golgi body or endoplasmic reticulum (Example 4).

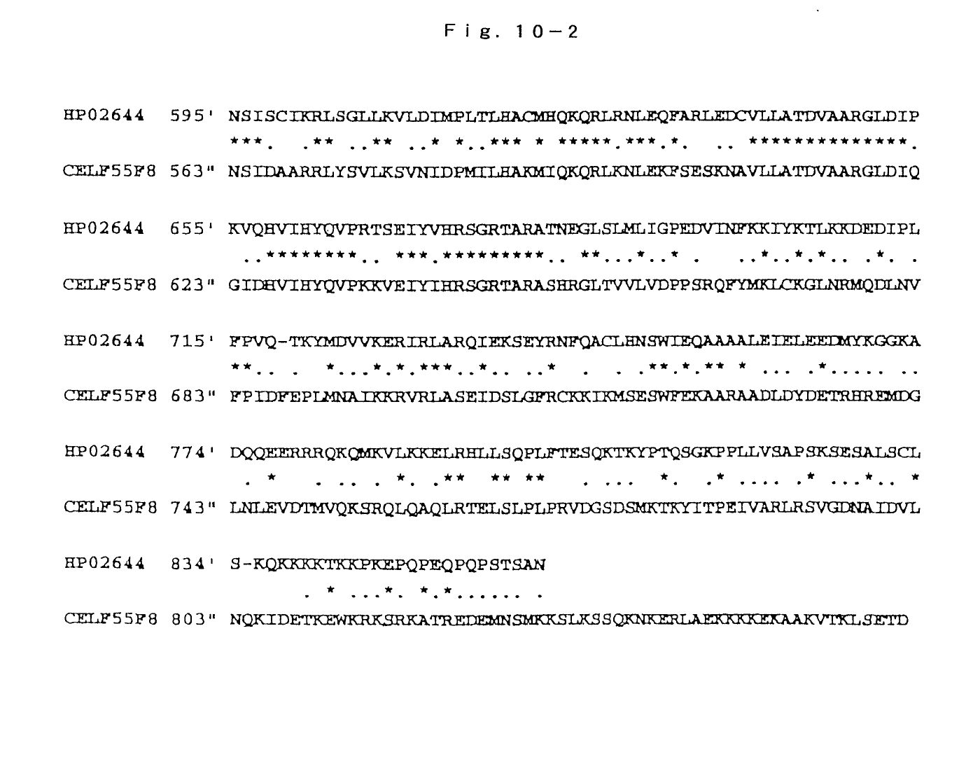

As a result of searching the protein database on the basis of the amino acid sequence of this protein, there was found a similarity to fission yeast putative ubiquinone biosynthesis methyltransferase (Accession No. CAB09781). FIG. 11 shows a comparison of the amino acid sequence between the human protein encoded by clone 20 and the fission yeast putative ubiquinone biosynthesis methyltransferase. In this figure, - means a gap, * means the same amino acid residue as the protein of this invention, and . means an amino acid residue similar to the protein of this invention, respectively. Over the whole region, they had a homology of 43.7%.

Further, as a result of referring to GenBank on the basis of the base sequence of cDNA of clone 20, those having a homology of not less than 90% (e.g. Accession No. AA338101) were found to have been registered in EST, but as it is of the partial sequence, it cannot be decided whether or not the same protein as that encoded by clone 20 is encoded.

21: HP10384

As the result of determining the total base sequence of cDNA insert of clone HP10384 obtained from human epidermal carcinoma cell line KB cDNA library, it was found that it had a structure of 126 bp 5′ untranslated region, 261 bp ORF and 350 bp 3′ untranslated region (SEQ ID No: 41). The ORF encoded the protein consisting of 86 amino acid residues (SEQ ID No: 42), and as a result of in vitro translation, a translated product of 10 kDa almost same as molecular weight 10,128 anticipated from the ORF was produced (Example 2). The fusion protein of this protein and GFP was found to be expressed in the whole cell or in the granular or coagulated mass form (Example 4).

Further, as a result of referring to GenBank on the basis of the base sequence of cDNA of clone 21, those having a homology of not less than 90% (e.g. Accession No. AF150406) were found to have been registered in EST, but as it is of the partial sequence, it cannot be decided whether or not the same protein as that encoded by clone 20 is encoded.

22: HP10431

As the result of determining the total base sequence of cDNA insert of clone HP10431 obtained from human liver cDNA library, it was found that it had a structure of 84 bp 5′ untranslated region, 537 bp ORF and 282 bp 3′ untranslated region (SEQ ID No: 43). The ORF encoded the protein consisting of 178 amino acid residues (SEQ ID No: 44), and as a result of in vitro translation, a translated product of 23 kDa slightly larger than molecular weight 20,277 anticipated from the ORF was produced (Example 2). The fusion protein of this protein and GFP was found to be expressed in the whole cell, and some of them was found in the granular or coagulated bulky form (Example 4).

Further, as a result of referring to GenBank on the basis of the base sequence of cDNA of clone 22, those having a homology of not less than 90% (e.g. Accession No. AW160991) were found to have been registered in EST, but as it is of the partial sequence, it cannot be decided whether or not the same protein as that encoded by clone 22 is encoded.

23: HP10437

As the result of determining the total base sequence of cDNA insert of clone HP10437 obtained from human stomach carcinoma cDNA library, it was found that it had a structure of 186 bp 5′ untranslated region, 354 bp ORF and 630 bp 3′ untranslated region (SEQ ID No: 45). The ORF encoded the protein consisting of 117 amino acid residues (SEQ ID No: 46), and as a result of in vitro translation, a translated product of 22 kDa larger than molecular weight 13,616 anticipated from the ORF was produced (Example 2). The fusion protein of this protein and GFP was found to be expressed in the whole cell or localized in the nucleus (Example 4).

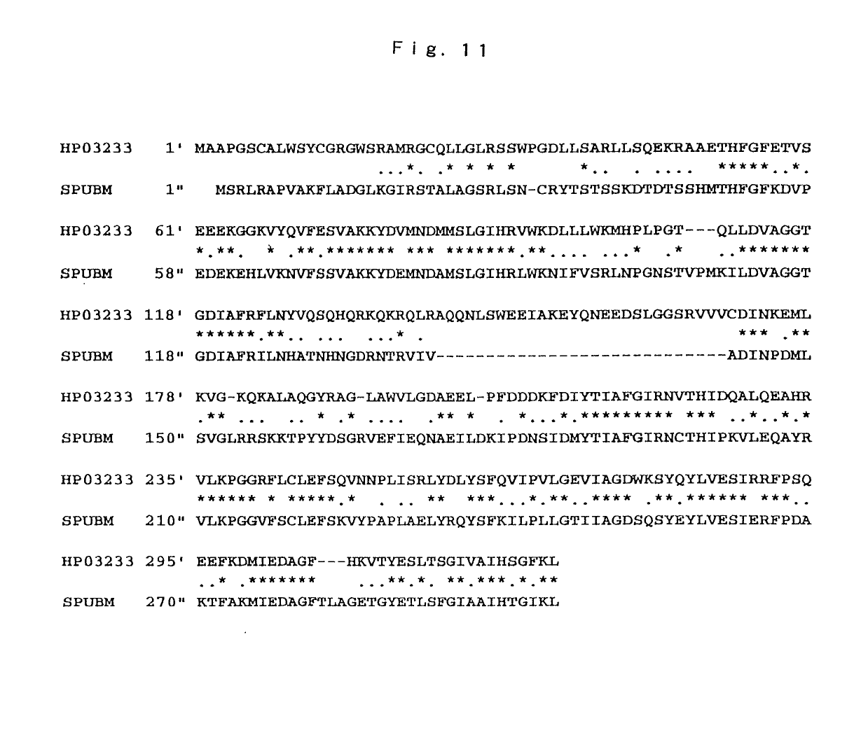

As a result of searching the protein database by using the amino acid sequence of this protein, there was found a similarity to human pp21 homologue (Accession No. AAF17229). FIG. 12 shows a comparison of the amino acid sequence between the human protein encoded by clone 23 and the human pp21 homologue. In this figure, - means a gap, * means the same amino acid residue as the protein of this invention, and . means an amino acid residue similar to the protein of this invention, respectively. Over the whole region, they had a homology of 39.4%. The pp21 is an analogue of transcription elongation factor SII.

Further, as a result of referring to GenBank on the basis of the base sequence of cDNA of clone 23, those having a homology of not less than 90% (e.g. Accession No. AA322053) were found to have been registered in EST, but as it is of the partial sequence, it cannot be decided whether or not the same protein as that encoded by clone 23 is encoded.

24: HP10525

As the result of determining the total base sequence of cDNA insert of clone HP10525 obtained from human stomach carcinoma DNA library, it was found that it had structure of 104 bp 5′ untranslated region, 261 bp ORF and 39 bp 3′ untranslated region (SEQ ID No: 47). The ORF encoded the protein consisting of 86 amino acid residues (SEQ ID No: 48), and as a result of in vitro translation, a translated product of 14 kDa slightly larger than molecular weight 10,110 anticipated from the ORF was produced (Example 2). The fusion protein of this protein and GFP was found to be expressed in the whole cell (Example 4).

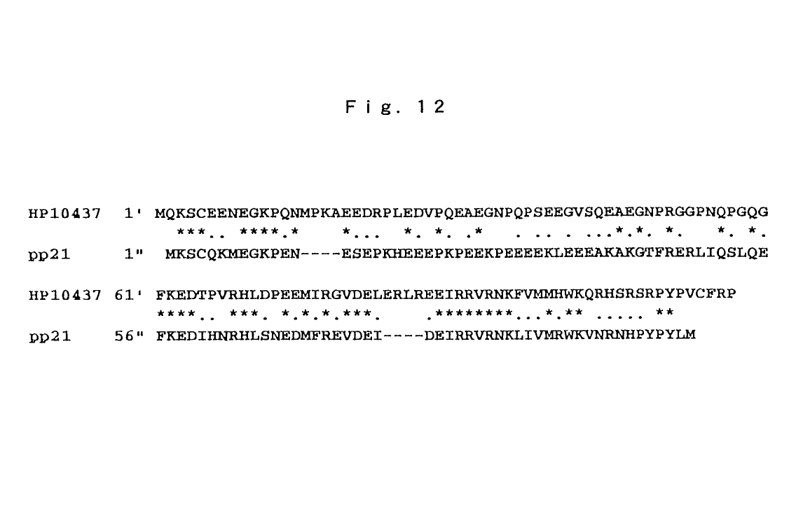

As a result of searching the protein database by using the amino acid sequence of this protein, there was a found similarity to fission yeast hypothetical protein SPAC8C9.11 (Accession No. AAC71096). FIG. 13 shows a comparison of the amino acid sequence between the human protein encoded by clone 24 and the fission yeast hypothetical protein SPAC8C9.11. In this figure, - means a gap, * means the same amino acid residue as the protein of this invention, and . means an amino acid residue similar to the protein of this invention, respectively. Over the whole region, they had a homology of 44.0%.

Further, as a result of referring to GenBank on the basis of the base sequence of cDNA of clone 24, those having a homology of not less than 90% (e.g. Accession No. AA310786) were found to have been registered in EST, but as it is of the partial sequence, it cannot be decided whether or not the same protein as that encoded by clone 24 is encoded.

25: HP10543

As the result of determining the total base sequence of cDNA insert of clone HP10543 obtained from human fibrosarcoma cell line HT-1080 cDNA library, it was found that it had a structure of 94 bp 5′ untranslated region, 540 bp ORF and 118 bp 3′ untranslated region (SEQ ID No: 49). The ORF encoded the protein consisting of 179 amino acid residues (SEQ ID No: 50), and as a result of in vitro translation, a translated product of 30 kDa larger than molecular weight 19,070 anticipated from the ORF was produced (Example 2). The fusion protein of this protein and GFP was found to be expressed in the whole cell or nucleus (Example 4).

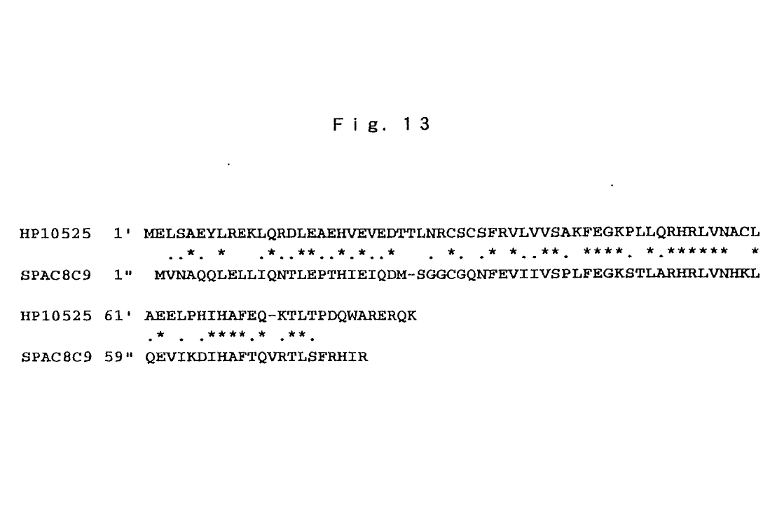

As a result of searching the protein database by using the amino acid sequence of this protein, there was found a similarity to mouse leucine-rich domain interacting protein 1 (Accession No. AAD17989). FIG. 14 shows a comparison of the amino acid sequence between the human protein encoded by clone 25 and the mouse leucine-rich domain interacting protein 1. In this figure, - means a gap, * means the same amino acid residue as the protein of this invention, and . means an amino acid residue similar to the protein of this invention, respectively. Over the C-terminal 138 amino acid residues, they had a homology of 69.6%.

Further, as a result of referring to GenBank on the basis of the base sequence of cDNA of clone 25, those having a homology of not less than 90% (e.g. Accession No. AA434567) were found to have been registered in EST, but as it is of the partial sequence, it cannot be decided whether or not the same protein as that encoded by clone 25 is encoded.

26: HP10565

As the result of determining the total base sequence of cDNA insert of clone HP10565 obtained from human stomach carcinoma cDNA library, it was found that it had a structure of 218p 5′ untranslated region, 570 bp ORF and 434 bp 3′ untranslated region (SEQ ID No: 51). The ORF encoded the protein consisting of 189 amino acid residues (SEQ ID No: 52), and as a result of in vitro translation, a translated product of 23 kDa slightly larger than molecular weight 20,663 anticipated from the ORF was produced (Example 2). The fusion protein of this protein and GFP was found to be localized in the Golgi body or endoplasmic reticulum (Example 4).

Further, as a result of referring to GenBank on the basis of the base sequence of cDNA of clone 26, those having a homology of not less than 90% (e.g. Accession No. AA258633) were found to have been registered in EST, but as it is of the partial sequence, it cannot be decided whether or not the same protein as that encoded by clone 26 is encoded.

27: HP10570

As the result of determining the total base sequence of cDNA insert of clone HP10570 obtained from human fibrosarcoma cell line HT-1080 cDNA library, it was found that it had a structure of 94 bp 5′ untranslated region, 354 bp ORF and 761 bp 3′ untranslated region (SEQ ID No: 53). The ORF encoded the protein consisting of 117 amino acid residues (SEQ ID No: 54), and as a result of in vitro translation, a translated product of 14 kDa almost same as molecular weight 12,767 anticipated from the ORF was produced (Example 2). The fusion protein of this protein and GFP was found to be expressed in the Golgi body or endoplasmic reticulum (Example 4).

Further, as a result of referring to GenBank on the basis of the base sequence of cDNA of clone 27, those having a homology of not less than 90% (e.g. Accession No. W07113) were found to have been registered in EST, but as it is of the partial sequence, it cannot be decided whether or not the same protein as that encoded by clone 27 is encoded.

28: HP03090

As the result of determining the total base sequence of cDNA insert of clone HP03090 obtained from human epidermal carcinoma cell line KB cDNA library, it was found that it had a structure of 25 bp 5′ untranslated region, 897 bp ORF and 841 bp 3′ untranslated region (SEQ ID No: 55). The ORF encoded the protein consisting of 298 amino acid residues (SEQ ID No: 56), and as a result of in vitro translation, a translated product of 34 kDa almost same as molecular weight 33,212 anticipated from the ORF was produced (Example 2). The fusion protein of this protein and GFP was found to be expressed in the whole cell (Example 4).

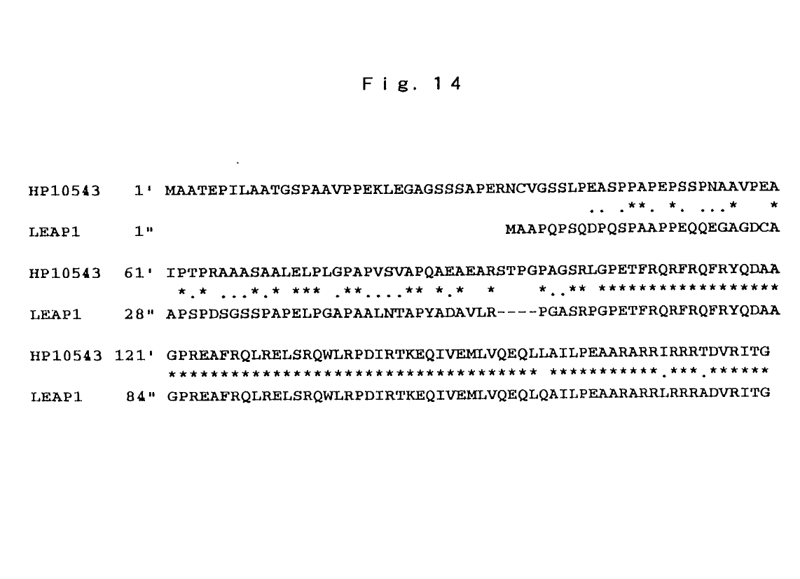

As a result of searching the protein database by using the amino acid sequence of this protein, there was found a similarity to nematoda hypothetical protein 32.0 kDa (Accession No. Q09253). FIG. 15 shows a comparison of the amino acid sequence between human protein encoded by clone 28 and nematoda hypothetical protein 32.0 kDa. In this figure, - means a gap, * means the same amino acid residue as the protein of this invention, and . means an amino acid residue similar to the protein of this invention, respectively. Over the whole region, they had a homology of 48.6%. Further, the C-terminal 292 amino acid residues starting from 7th leucine of this protein was found to coincide with the C-terminal amino acid residues starting from the 213rd leucine of human CGI-150 protein (Accession No. AAD34145).

Further, as a result of referring to GenBank on the basis of the base sequence of cDNA of clone 28, those having a homology of not less than 90% (e.g. Accession No. H06942) were found to have been registered in EST, but as it is of the partial sequence, it cannot be decided whether or not the same protein as that encoded by clone 28 is encoded.

29: HP03115

As the result of determining the total base sequence of cDNA insert of clone HP03115 obtained from human epidermal carcinoma cell line KB cDNA library, it was found that it had a structure of 302 bp 5′ untranslated region, 1077 bp ORF and 534 bp 3′ untranslated region (SEQ ID No: 57). The ORF encoded the protein consisting of 358 amino acid residues (SEQ ID No: 58), and as a result of in vitro translation, a translated product of 39 kDa almost same as molecular weight 40,275 anticipated from the ORF was produced (Example 2). The fusion protein between this protein and GFP was found to be expressed in the granular form the cytoplasm (Example 4).

In the amino acid sequence of this protein, there was found the C3HC4 type zinc finger (RING finger) motif (from 42nd cysteine to 51st alanine).

Further, as a result of referring to GenBank on the basis of the base sequence of cDNA of clone 29, those having a homology of not less than 90% (e.g. Accession No. AA428229) were found to have been registered in EST, but as it is of the partial sequence, it cannot be decided whether or not the same protein as that encoded by clone 29 is encoded.

30: HP03145

As the result of determining the total base sequence of cDNA insert of clone HP03145 obtained from human epidermal carcinoma cell line KB cDNA library, a it was found that it had structure of 31 bp 5′ untranslated region, 1116 bp ORF and 373 bp 3′ untranslated region (SEQ ID No: 59). The ORF encoded the protein consisting of 371 amino acid residues (SEQ ID No: 60), and as a result of in vitro translation, a translated product of 41 kDa almost same as molecular weight 40,463 anticipated from the ORF was produced (Example 2). The fusion protein of this protein and GFP was found to be localized in the Golgi body or endoplasmic reticulum (Example 4).

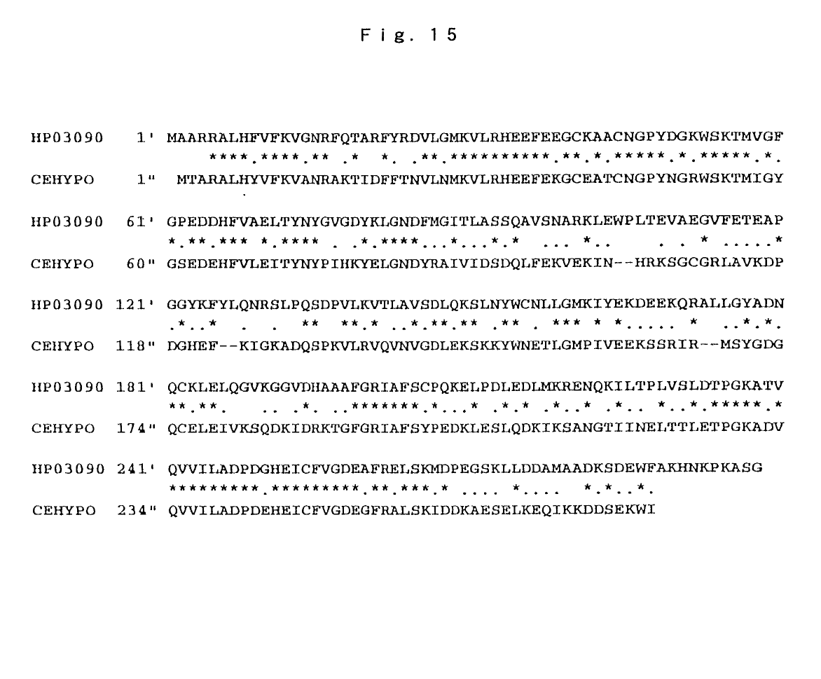

As a result of searching the protein database by using the amino acid sequence of this protein, there was found a similarity to fission yeast mitochondrial p-hydroxybenzoate polyprenyltransferase-like protein (Accession No. Q10252). FIG. 16 shows a comparison of the amino acid sequence between the human protein encoded by clone 30 and the fission yeast mitochondria parahydroxybenzoate polyprenyltransferase-like protein. In this figure, - means a gap, * means the same amino acid residue as the protein of this invention, and . means an amino acid residue similar to the protein of this invention, respectively. Over the whole region except for the N-terminal, they had a homology of 46.4%.

Moreover, the 120 amino acid residues from 198th methionine to 317th glutamine in this protein conincided with the N-terminal amino acid residues of human hypothetical protein (Accession No. AAC72955).

Further, as a result of referring to GenBank on the basis of the base sequence of cDNA of clone 30, those having a homology of not less than 90% (e.g. Accession No. N94036) were found to have been registered in EST, but as it is of the partial sequence, it cannot be decided whether or not the same protein as that encoded by clone 30 is encoded.

31: HP03185

As the result of determining the total base sequence of cDNA insert of clone HP03185 obtained from human fibrosarcoma cell line HT-1080 cDNA library, it was found that it had a structure of 182 bp 5′ untranslated region, 1119 bp ORF and 430 bp 3′ untranslated region (SEQ ID No: 61). The ORF encoded the protein consisting of 372 amino acid residues (SEQ ID No: 62), and as a result of in vitro translation, a translated product of 44 kDa slightly larger than molecular weight 40,033 anticipated from the ORF was produced (Example 2). The fusion protein of this protein and GFP was found to be localized in the nucleus or nucleolus (Example 4).

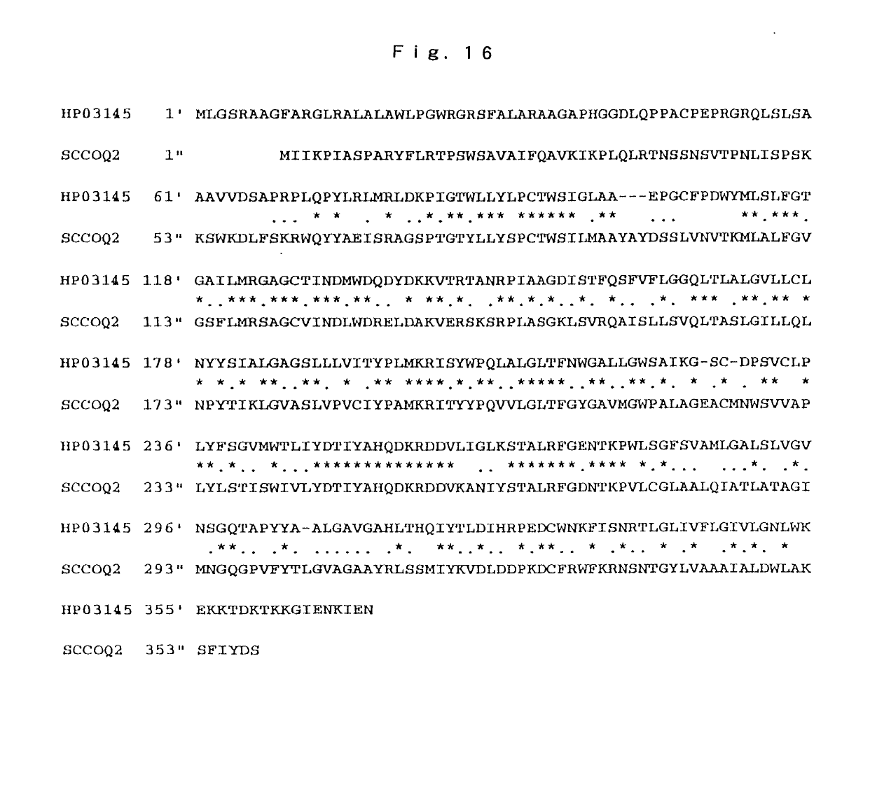

As a result of searching the protein database by using the amino acid sequence of this protein, there was found a similarity to human bistone macroH2A1.2 (Accession No. AAC33433). FIG. 17 shows a comparison of the amino acid sequence between the human protein encoded by clone 31 and the human histone macroH2A1.2. In this figure, - means a gap, * means the same amino acid residue as the protein of this invention, and . means an amino acid residue similar to the protein of this invention, respectively. Over the whole region, they had a homology of 67.5%. Further, the histone forming a complex with DNA participates in regulating the gene expression.

Further, as a result of referring to GenBank on the basis of the base sequence of cDNA of clone 31, those having a homology of not less than 90% (e.g. Accession No. AI 878933) were found to have been registered in EST, but as it is of the partial sequence, it cannot be decided whether or not the same protein as that encoded by clone 31 is encoded.

32: HP03324

As the result of determining the total base sequence of cDNA insert of clone HP03324 obtained from human lymphoma cell line U937 cDNA library, it was found that it had a structure of 20 bp 5′ untranslated region, 678 bp ORF and 212 bp 3′ untranslated region (SEQ ID No: 63). The ORF encoded the protein consisting of 225 amino acid residues (SEQ ID No: 64), and as a result of in vitro translation, a translated product of 25 kDa almost same as molecular weight 24,415 anticipated from the ORF was produced (Example 2). The fusion protein of this protein and GFP was found to be localized in the mitochondria (Example 4).

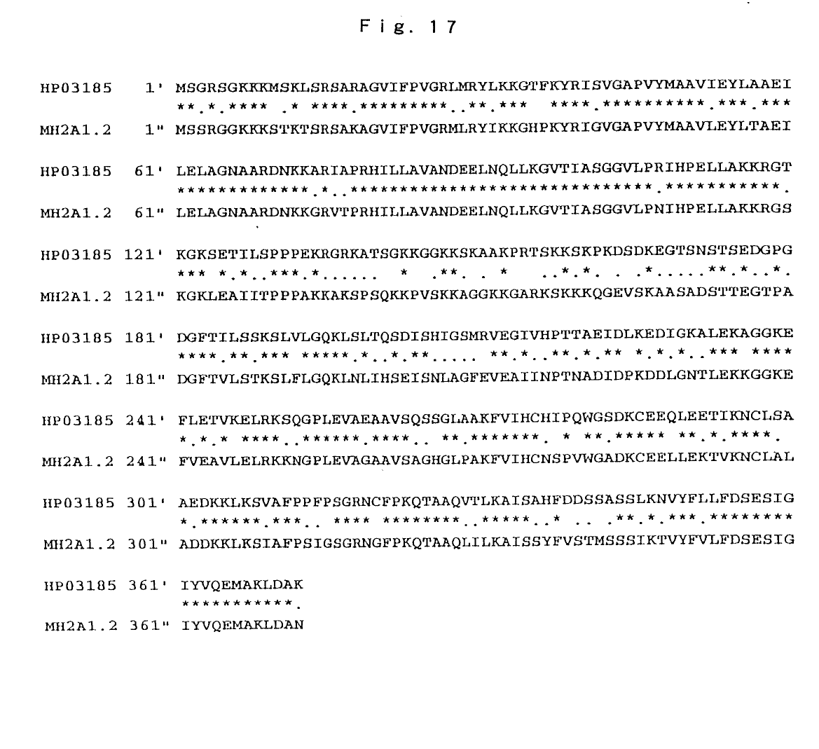

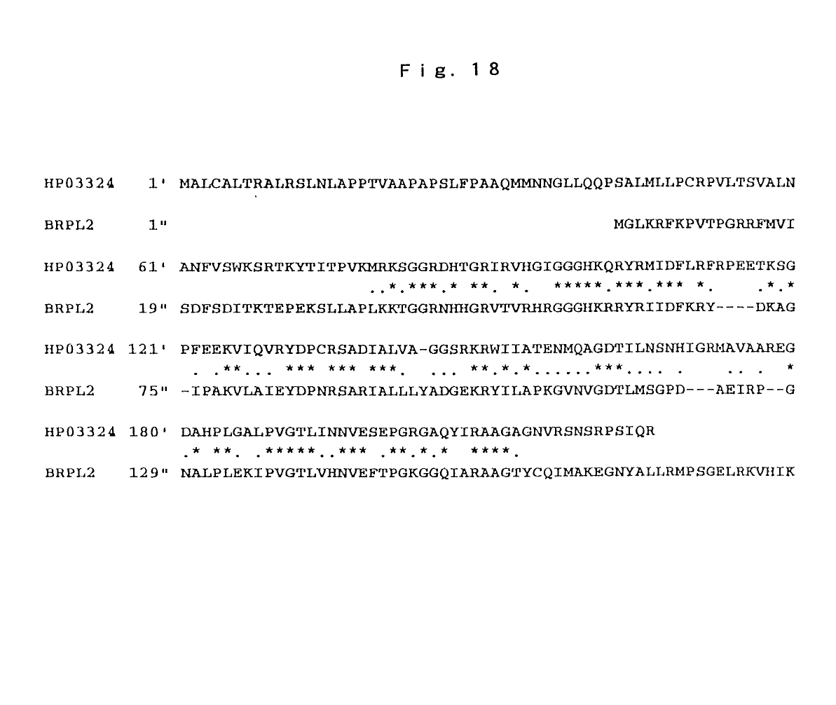

As a result of searching the protein database by using the amino acid sequence of this protein, there was found a similarity to bacteria ribosomal protein L2 (Accession No. AAD36563). FIG. 18 shows a comparison of the amino acid sequence between the human protein encoded by clone 32 and the bacteria ribosomal protein L2. In this figure, - means a gap, * means the same amino acid residue as the protein of this invention, and . means an amino acid residue similar to the protein of this invention, respectively. Over the intermediary 135 amino acid residues, they had a homology of 44.4%. Further, the N-terminal 211 amino acid residues of this protein were found to show a homology of 99.1% with the N-terminal amino acid residues of human CGI-22 protein (Accession No. AAD27731).

Further, as a result of referring to GenBank on the basis of the base sequence of cDNA of clone 32, those having a homology of not less than 90% (e.g. Accession No. R72376) were found to have been registered in EST, but as it is of the partial sequence, it cannot be decided whether or not the same protein as that encoded by clone 32 is encoded.

33: HP10052

As the result of determining the total base sequence of cDNA insert of clone HP10052 obtsained from human fibrosarcoma cell line HT-1080 cDNA library, it was found that it had a structure of 155 bp 5′ untranslated region, 345 bp ORF and 284 bp 3′ untranslated region (SEQ ID No: 65). The ORF encoded the protein consisting of 114 amino acid residues (SEQ ID No: 66), and as a result of in vitro translation, a translated product of 17 kDa larger than molecular weight 11,770 anticipated from ORF was produced (Example 2). The fusion protein of this protein and GFP was found to be expressed in the whole cell (Example 4).

Further, as a result of referring to GenBank on the basis of the base sequence of cDNA of clone 33, those having a homology of not less than 90% (e.g. Accession No. AI 815489) were found to have been registered in EST, but as it is of the partial sequence, it cannot be decided whether or not the same protein as that encoded by clone 33 is encoded.

34: HP10626

As the result of determining the total base sequence of cDNA insert of clone HP10626 obtained from human epidermal carcinoma cell line KB cDNA library, it was found that it had a structure of 347 bp 5′ untranslated region, 423 bp ORF and 214 bp 3′ untranslated region (SEQ ID No: 67). The ORF encoded the protein consisting of 140 amino acid residues (SEQ ID No: 68), and as a result of in vitro translation, a translated product of 14 kDa almost same as molecular weight 14,555 anticipated from the ORF was produced (Example 2). The fusion protein of this protein and GFP was found to be expressed in the nucleus (Example 4).

Further, as a result of referring to GenBank on the basis of the base sequence of cDNA of clone 34, those having a homology of not less than 90% (e.g. Accession No. AA234649) were found to have been registered in EST, but as it is of the partial sequence, it cannot be decided whether or not the same protein as that encoded by clone 34 is encoded.

35: HP10633

As the result of determining the total base sequence of cDNA insert of clone HP10633 obtained from human fibrosarcoma cell line HT-1080 cDNA library, it was found that it had a structure of 356 bp 5′ untranslated region, 258 bp ORF and 250 bp 3′ uintranslated region (SEQ ID No: 69). The ORF encoded the protein consisting of 85 amino acid residues (SEQ ID No: 70), and as a result of in vitro translation, a translated product of 10 kDa almost same as molecular weight 9,771 anticipated from the ORF was produced (Example 2). The fusion protein of this protein and GFP was found to be expressed in the whole cell (Example 4).

Further, as a result of referring to GenBank on the basis of the base sequence of cDNA of clone 35, those having a homology of not less than 90% (e.g. Accession No. R73005) were found to have been registered in EST, but as it is of the partial sequence, it cannot be decided whether or not the same protein as that encoded by clone 35 is encoded.

36: HP10637

As the result of determining the total base sequence of cDNA insert of clone HP10637 obtained from human fibrosarcoma cell line HT1080 cDNA library, it was found that it had a structure of 120 bp 5′ untranslated region, 1740 bp ORF and 757 bp 3′ untranslated region (SEQ ID No: 71). The ORF encoded the protein consisting of 579 amino acid residues (SEQ ID No: 72). The fusion protein of this protein and GFP was found to be expressed in the whole cell, and some of them was found in the form of particle coagulated mass (Example 4).

Further, as a result of referring to GenBank on the basis of the base sequence of cDNA of clone 36, those having a homology of not less than 90% (e.g. Accession No. AI 929698) were found to have been registered in EST, but as it is of the partial sequence, it cannot be decided whether or not the same protein as that encoded by clone 36 is encoded.

37: HP10648

As the result of determining the total base sequence of cDNA insert of clone HP10648 obtained from human epidermal carcinoma cell line KB cDNA library, it was found that it had a structure of 38 bp 5′ untranslated region, 1083 bp ORF and 689 bp 3′ untranslated region (SEQ ID No: 73). The ORF encoded the protein consisting of 360 amino acid residues (SEQ ID No: 74), and as a result of in vitro translation, a translated product of 50 kDa larger than molecular weight 40, 211 anticipated from the ORF was produced (Example 2). The fusion protein of this protein and GFP was found to be localized in the nucleus (Example 4).

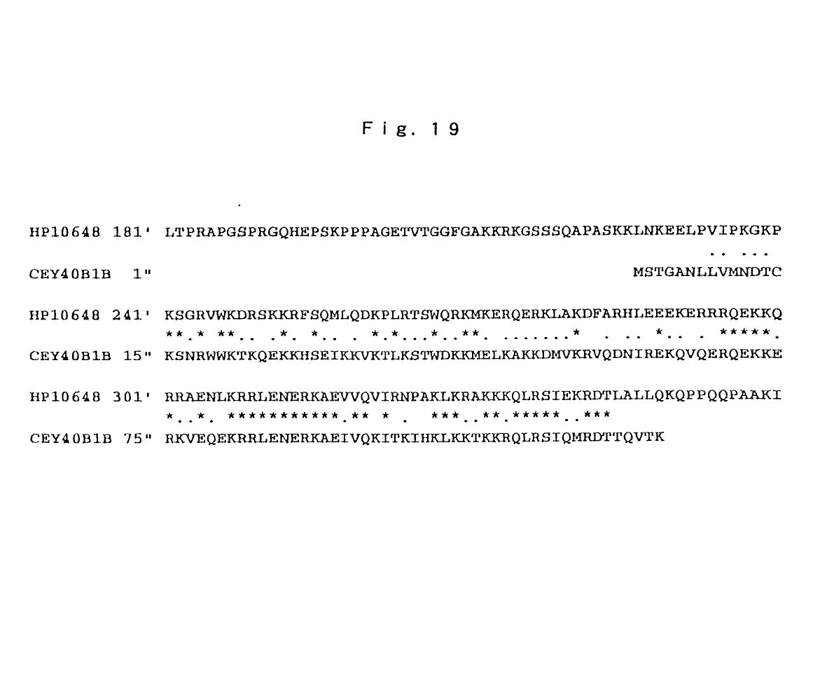

As a result of searching the protein database by using the amino acid sequence of this protein, there was found a similarity to nematoda hypothetical protein Y40B1B.7 (Accession No. CAA21606). FIG. 19 shows a comparison of the amino acid sequence between the human protein encoded by clone 37 and the nematoda hypothetical protein Y40B1B.7. In this figure, - means a gap, * means the same amino acid residue as the protein of this invention, and . means an amino acid residue similar to the protein of this invention, respectively. Over the C-terminal 111 amino acid residues, they had a homology of 43.2%.

Further, as a result of referring to GenBank on the basis of the base sequence of cDNA of clone 37, those having a homology of not less than 90% (e.g. Accession No. W39612) were found to have been registered in EST, but as it is of the partial sequence, it cannot be decided whether or not the same protein as that encoded by clone 37 is encoded.

38: HP10211

As the result of determining the total base sequence of cDNA insert of clone HP10211 obtained from human osteosarcoma cell line Saos-2 cDNA library, it was found that it had a structure of 216p 5′ untranslated region, 381 bp ORF and 1023 bp 3′ untranslated region (SEQ ID No: 75). The ORF encoded the protein consisting of 126 amino acid residues (SEQ ID No: 76), and as a result of in vitro translation, a translated product of 14 kDa slightly larger than molecular weight 12,758 anticipated from the ORF was produced (Example 2). The fusion protein of this protein and GFP was found to be expressed in the whole cell (Example 4).

Further, as a result of referring to GenBank on the basis of the base sequence of cDNA of clone 38, those having a homology of not less than 90% (e.g. Accession No. D81861) were found to have been registered in EST, but as it is of the partial sequence, it cannot be decided whether or not the same protein as that encoded by clone 38 is encoded.

39: HP10332

As the result of determining the total base sequence of cDNA insert of clone HP10332 obtained from human stomach carcinoma cDNA library, it was found that it had a structure of 184 bp 5′ untranslated region, 858 bp ORF and 307 bp 3′ untranslated region (SEQ ID No: 77). The ORF encoded the protein consisting of 285 amino acid residues (SEQ ID No: 78), and as a result of in vitro translation, a translated product of 35 kDa slightly larger than molecular weight 32,158 anticipated from the ORF was produced (Example 2). The fusion protein of this protein and GFP was found to be expressed in the whole cell, but some cells were expressed in the Golgi body or endoplasmic reticulum (Example 4).

Further, as a result of referring to GenBank on the basis of the base sequence of cDNA of clone 39, those having a homology of not less than 90% (e.g. Accession No. AA025985) were found to have been registered in EST, but as it is of the partial sequence, it cannot be decided whether or not the same protein as that encoded by clone 39 is encoded.

40: HP10641

As the result of determining the total base sequence of cDNA insert of clone HP10641 obtained from human epidermal carcinoma cell line KB cDNA library, it was found that it had a structure of 78 bp 5′ untranslated region, 990 bp ORF and 287 bp 3) untranslated region (SEQ ID No: 79). The ORF encoded the protein consisting of 329 amino acid residues (SEQ ID No: 80), and as a result of in vitro translation, a translated product of 42 kDa larger than molecular weight 36,537 anticipated from the ORF was produced (Example 2). The fusion protein of this protein and GFP was found to be expressed in the whole cell (Example 4).

Further, as a result of referring to GenBank on the basis of the base sequence of cDNA of clone 40, those having a homology of not less than 90% (e.g. Accession No. T09308) were found to have been registered in EST, but as it is of the partial sequence, it cannot be decided whether or not the same protein as that encoded by clone 40 is encoded. Moreover, a clone (Accession No. AF 161491) showing a homology of 99.9% was found to have been registered, but this clone encodes a protein different from that of clone 40 because it brings about a frame shift due to shortage of G corresponding to 865th of clone 40.

41: HP10650

As the result of determining the total base sequence of cDNA insert of clone HP10650 obtained from human epidermal carcinoma cell line KB cDNA library, it was found that it had a structure of 28 bp 5′ untranslated region, 702 bp ORF and 813 bp 3′ untranslated region (SEQ ID No: 81). The ORF encoded the protein consisting of 233 amino acid residues (SEQ ID No: 82), and as a result of in vitro translation, a translated product of 30 kDa larger than molecular weight 25,846 anticipated from the ORF was produced (Example 2). The fusion protein of this protein and GFP was found to be expressed in the particle form in the cytoplasm (Example 4).

Further, as a result of referring to GenBank on the basis of the base sequence of cDNA of clone 41, those having a homology of not less than 90% (e.g. Accession No. AA494499) were found to have been registered in EST, but as it is of the partial sequence, it cannot be decided whether or not the same protein as that encoded by clone 41 is encoded.

42: HP10654

As the result of determining the total base sequence of cDNA insert of clone HP10654 obtained from human epidermal carcinoma cell line KB cDNA library, it was found that it had a structure of 30 bp 5′ untranslated region, 552 bp ORF and 854 bp 3′ untranslated region (SEQ ID No: 83). The ORF encoded the protein consisting of 183 amino acid residues (SEQ ID No: 84), and as a result of in vitro translation, a translated product of 24 kDa slightly larger than molecular weight 21,077 anticipated from the ORF was produced (Example 2). The fusion protein of this protein and GFP was found to be expressed in the whole cell (Example 4).

Further, as a result of referring to GenBank on the basis of the base sequence of cDNA of clone 42, those having a homology of not less than 90% (e.g. Accession No. AA459480) were found to have been registered in EST, but as it is of the partial sequence, it cannot be decided whether or not the same protein as that encoded by clone 42 is encoded.

43: HP10657

As the result of determining the total base sequence of cDNA insert of clone HP10657 obtained from human lymphoma cell line U937 cDNA library, it was found that it had a structure of 101 bp 5′ untranslated region, 1143 bp ORF and 113 bp 3′ untranslated region (SEQ ID No: 85). The ORF encoded the protein consisting of 380 amino acid residues (SEQ ID No: 86), and as a result of in vitro translation, a translated product of 41 kDa almost same as molecular weight 40,485 anticipated from the ORF was produced (Example 2). The fusion protein of this protein and GFP was found to be expressed in the whole cell (Example 4).

Further, as a result of referring to GenBank on the basis of the base sequence of cDNA of clone 43, those having a homology of not less than 90% (e.g. Accession No. R25280) were found to have been registered in EST, but as it is of the partial sequence, it cannot be decided whether or not the same protein as that encoded by clone 43 is encoded.

44: HP10659

As the result of determining the total base sequence of cDNA insert of clone HP10659 obtained from human lymphoma cell line U937 cDNA library, it was found that it had a structure of 73 bp 5′ untranslated region, 783 bp ORF and 543 bp 3′ untranslated region (SEQ ID No: 87). The ORF encoded the protein consisting of 260 amino acid residues (SEQ ID No: 88), and as a result of in vitro translation, a translated product of 31 kDa almost same as molecular weight 30, 815 anticipated from the ORF was produced (Example 2). The fusion protein of this protein and GFP was found to be localized in the bulky coagulated mass or granular form in the cytoplasm (Example 4).

Further, as a result of referring to GenBank on the basis of the base sequence of cDNA of clone 44, those having a homology of not less than 90% (e.g. Accession No. AA356158) were found to have been registered in EST, but as it is of the partial sequence, it cannot be decided whether or not the same protein as that encoded by clone 44 is encoded.

45: HP10681

As the result of determining the total base sequence of cDNA insert of clone HP10681 obtained from human fibrosarcoma cell line HT-1080 cDNA library, it was found that it had a structure of 151 bp 5′ untranslated region, 825 bp ORF and 143 bp 3′ untranslated region (SEQ ID No: 89). The ORF encoded the protein consisting of 274 amino acid residues (SEQ ID No: 90), and as a result of in vitro translation, a translated product of 32 kDa almost same as molecular weight 31,045 anticipated from the ORF was produced (Example 2). The fusion protein of this protein and GFP was found to be expressed in the whole cell, and some of them were expressed also in the granular form (Example 4).

Further, as a result of referring to GenBank on the basis of the base sequence of cDNA of clone 45, those having a homology of not less than 90% (e.g. Accession No. AA406451) were found to have been registered in EST, but as it is of the partial sequence, it cannot be decided whether or not the same protein as that encoded by clone 45 is encoded.

46: HP10077

As the result of determining the total base sequence of cDNA insert of clone HP10077 obtained from human stomach carcinoma cDNA library, it was found that it had a structure of 132 bp 5′ untranslated region, 306 bp ORF and 102 bp 3′ untranslated region (SEQ ID No: 91). The ORF encoded the protein consisting of 101 amino acid residues (SEQ ID No: 92), and as a result of in vitro translation, a translated product of 11 kDa almost same as molecular weight 11,521 anticipated from the ORF was produced (Example 2). The fusion protein of this protein and GFP was found to be expressed in the whole cell (Example 4).

Further, as a result of referring to GenBank on the basis of the base sequence of cDNA of clone 46, those having a homology of not less than 90% (Accession No. AF086207) were found to have been registered in EST, but it is of the complementary sequence, it doesn't encode a protein. Moreover, those having a homology of not less than 90% (e.g. Accession No. W48698 or AF086207) were found to have been registered in the EST, but it cannot be decided whether or not the same protein as that encoded by clone 46 is encoded.

47: HP10162

As the result of determining the total base sequence of cDNA insert of clone HP10162 obtained from human osteosarcoma cell line Saos-2 cDNA library, it was found that it had a structure of 32 bp 5′ untranslated region, 837 bp ORF and 190 bp 3′ untranslated region (SEQ ID No: 93). The ORF encoded the protein consisting of 278 amino acid residues (SEQ ID No: 94), and as a result of in vitro translation, a translated product of 32 kDa almost same as molecular weight 31,844 anticipated from the ORF was produced (Example 2). The fusion protein of this protein and GFP was found as the granular form in the whole cell (Example 4).

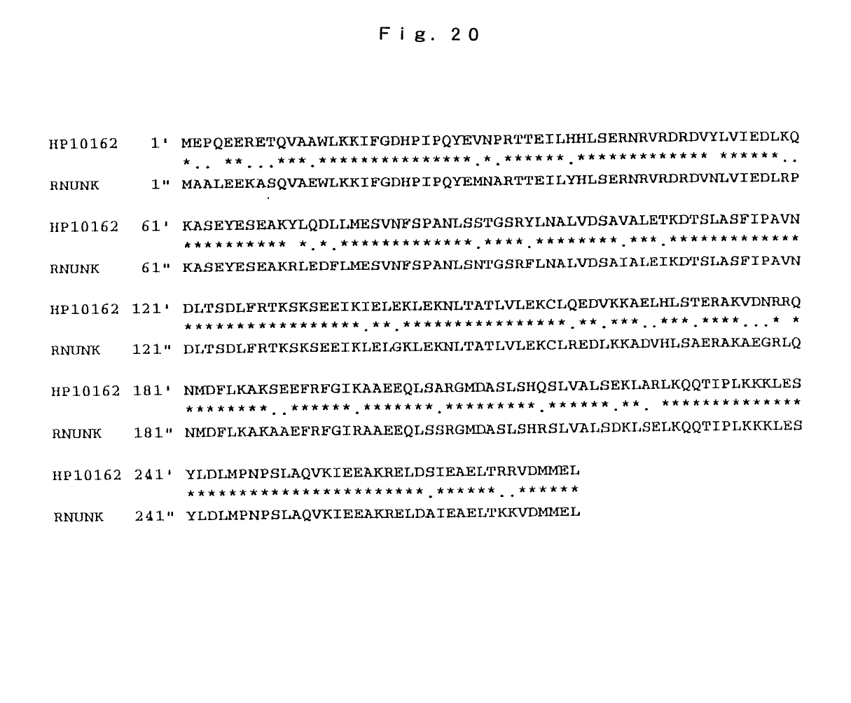

As a result of searching the protein database by using the amino acid sequence of this protein, there was found a similarity to rat hypothetical protein (Accession No. AAF00052). FIG. 20 shows a comparison of the amino acid sequence between the human protein encoded by clone 47 and rat hypothetical protein. In this figure, - means a gap, * means the same amino acid residue as the protein of this invention, and . means an amino acid residue similar to the protein of this invention, respectively. Over the whole region, they had a homology of 84.9%.

Further, as a result of referring to GenBank on the basis of the base sequence of cDNA of clone 47, those having a homology of not less than 90% (e.g. Accession No. AA377040) were found to have been registered in EST, but as it is of the partial sequence, it cannot be decided whether or not the same protein as that encoded by clone 47 is encoded.

48: HP10334

As the result of determining the total base sequence of cDNA insert of clone HP10334 obtained from human fibrosarcoma cell line HT-1080 cDNA library, it was found that it had a structure of 102 bp 5′ untranslated region, 282 bp ORF and 398 bp 3′ untranslated region (SEQ ID No: 95). The ORF encoded the protein consisting of 93 amino acid residues (SEQ ID No: 96), and as a result of in vitro translation, a translated product of 14 kDa slightly larger than molecular weight 10,431 anticipated from the ORF was produced (Example 2). The fusion protein of this protein and GFP was found to be expressed in the whole cell (Example 4).

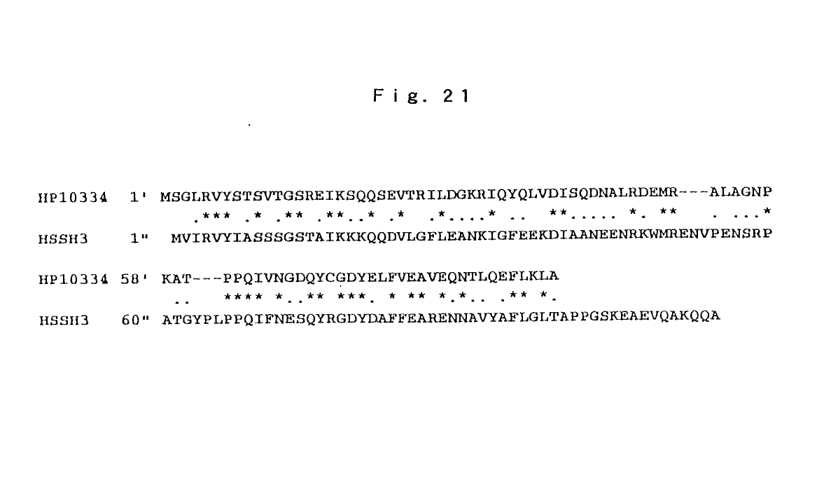

As a result of searching the protein database by using the amino acid sequence of this protein, there was found a similarity to human SH3 domain binding glutamic acid-rich-like protein (Accession No. NP 003013). FIG. 21 shows a comparison of the amino acid sequence between the human protein encoded by clone 48 and the human SH3 domain binding glutamic acid-rich-like protein. In this figure, - means a gap, * means the same amino acid residue as the protein of this invention, and . means an amino acid residue similar to the protein of this invention, respectively. Over the whole region, the had a homology of 37.5%.

Further, as a result of referring to GenBank on the basis of the base sequence of cDNA of clone 48, those having a homology of not less than 90% (e.g. Accession No. AA299350) were found to have been registered in EST, but as it is of the partial sequence, it cannot be decided whether or not the same protein as that encoded by clone 48 is encoded.

49: HP10400

As the result of determining the total base sequence of cDNA insert of clone HP10400 obtained from human stomach carcinoma cDNA library, it was found that it had a structure of 21 bp 5′ untranslated region, 174 bp ORF and 222 bp 3′ untranslated region (SEQ ID No: 97). The ORF encoded the protein consisting of 57 amino acid residues (SEQ ID No: 98), and as a result of in vitro translation, a translated product of 8 kDa slightly larger than molecular weight 6,207 anticipated from the ORF was produced (Example 2). The fusion protein of this protein and GFP was found to be expressed in the whole cell (Example 4).

Further, as a result of referring to GenBank on the basis of the base sequence of cDNA of clone 49, those having a homology of not less than 90% (e.g. Accession No. W05345) were found to have been registered in EST, but as it is of the partial sequence, it cannot be decided whether or not the same protein as that encoded by clone 49 is encoded.

50: HP10410

As the result of determining the total base sequence of cDNA insert of clone HP10410 obtained from human stomach carcinoma cDNA library, it was found that it had a structure of 64 bp 5′ untranslated region, 348 bp ORF and 285 bp 3′ untranslated region (SEQ ID No: 99). The ORF encoded the protein consisting of 115 amino acid residues (SEQ ID No: 100), and as a result of in vitro translation, a translated product of 14 kDa larger than molecular weight 12,506 anticipated from the ORF was produced (Example 2). The fusion protein of this protein and GFP was found to be expressed in the cytoplasm and nucleus (Example 4).

Further, as a result of referring to GenBank on the basis of the base sequence of cDNA of clone 50, those having a homology of not less than 90% (e.g. Accession No. T87538) were found to have been registered in EST, but as it is of the partial sequence, it cannot be decided whether or not the same protein as that encoded by clone 50 is encoded.

51: HP10417

As the result of determining the total base sequence of cDNA insert of clone HP10417 obtained from human stomach carcinoma cDNA library, it was found that it had a structure of 461 bp 5′ untranslated region, 333 bp ORF and 710 bp 3′ untranslated region (SEQ ID No: 101). The ORF encoded the protein consisting of 110 amino acid residues (SEQ ID No: 102), and as a result of in vitro translation, a translated product of 14 kDa slightly larger than molecular weight 11,667 anticipated from the ORF was produced (Example 2). The fusion protein of this protein and GFP was found to be expressed in the whole cell (Example 4).

Further, as a result of referring to GenBank on the basis of the base sequence of cDNA of clone 51, those having a homology of not less than 90% (e.g. Accession No. C15811) were found to have been registered in EST, but as it is of the partial sequence, it cannot be decided whether or not the same protein as that encoded by clone 51 is encoded.

52: HP10482

As the result of determining the total base sequence of cDNA insert of clone HP10482 obtained from human fibrosarcoma cell line HT-1080 cDNA library, it was found that it had a structure of 123 bp 5′ untranslated region, 402 bp ORF and 521 bp 3′ untranslated region (SEQ ID No: 103). The ORF encoded the protein consisting of 133 amino acid residues (SEQ ID No: 104), and as a result of in vitro translation, a translated product in high molecular weight was produced (Example 2). The fusion protein of this protein and GFP was found to be expressed in the whole cell (Example 4).

As a result of referring to GenBank on the basis of the base sequence of cDNA of clone 52, it had a homology with complementary sequence of profilaggrin (e.g. Accession No. M60499). Further, those having a homology of not less than 90% (e.g. Accession No. M62201) were found to have been registered in EST, but as it is of the partial sequence, it cannot be decided whether or not the same protein as that encoded by clone 52 is encoded.

53: HP10499

As the result of determining the total base sequence of cDNA insert of clone HP10499 obtained from human stomach carcinoma cDNA library, considering 79th TGA as selenocysteine but not as a stop codon, it was found that it had a structure of 54 bp 5′ untranslated region, 207 bp ORF and 80 bp 3′ untranslated region (SEQ ID No: 105). The ORF encoded the protein consisting of 68 amino acid residues (SEQ ID No: 106). The fusion protein of this protein and GFP was expressed by fusing GFP cDNA just before 259th stop codon of this ORF, it was found in the whole cell (Example 4). Since the fusion protein had been expressed in spite of the initiating codon in this ORF being not found except in 55th ATG only, 79th TGA is considered to encode selenocysteine without functioning as a stop codon.

Further, as a result of referring to GenBank on the basis of the base sequence of cDNA of clone 53, those having a homology of not less than 90% (e.g. Accession No. AA523172) were found to have been registered in EST, but as it is of the partial sequence, it cannot be decided whether or not the same protein as that encoded by clone 53 is encoded.

54: HP10522

As the result of determining the total base sequence of cDNA insert of clone HP10522 obtained from human stomach carcinoma cDNA library, it was found that it had a structure of 12 bp 5′ untranslated region, 999 bp ORF and 673 bp 3′ untranslated region (SEQ ID No: 107). The ORF encoded the protein consisting of 332 amino acid residues (SEQ ID No: 108). As a result of in vitro translation, a translated product of 41 kDa slightly larger than molecular weight 37,512 expected from the ORF was produced (Example2). The fusion protein of this protein and GFP was localized in the mitochondria (Example 4).

As a result of referring to GenBank on the basis of the base sequence of cDNA of clone 54, those having a homology of not less than 90% (e.g. Accession No. C03423) were found to have been registered in EST, but as it is of the partial sequence, it cannot be decided whether or not the same protein as that encoded by clone 54 is encoded.

55: HP10532

As the result of determining the total base sequence of cDNA insert of clone HP10532 obtained from human stomach carcinoma cDNA library, it was found that it had a structure of 80 bp 5′ untranslated region, 480 bp ORF and 167 bp 3′ untranslated region (SEQ ID No: 109). The ORF encoded the protein consisting of 159 amino acid residues (SEQ ID No: 110). The fusion protein of this protein and GFP was expressed in the whole cell (Example 4).

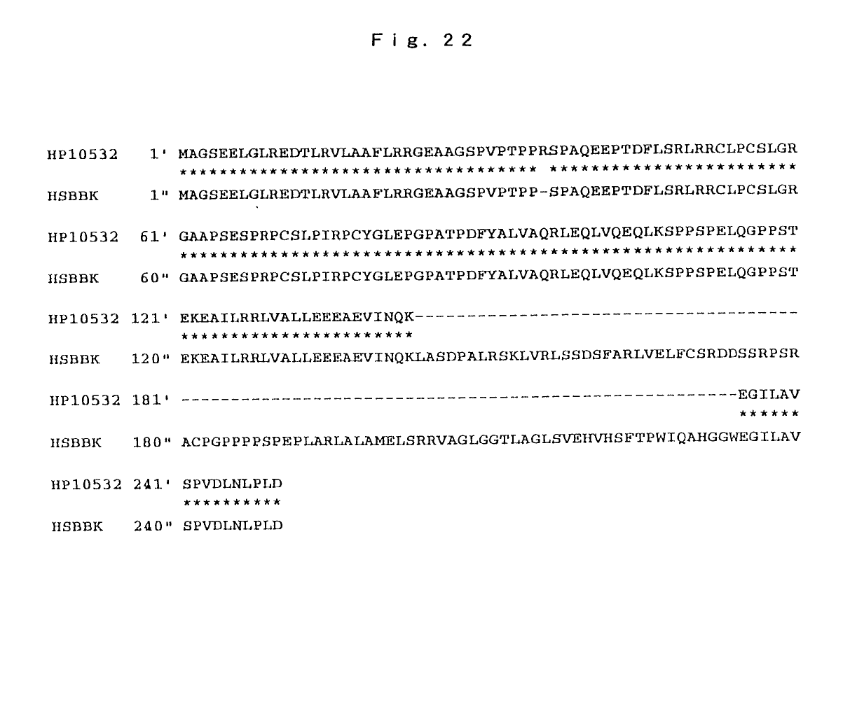

As a result of searching the protein database by using the amino acid sequence of this protein, there was found a similarity to human apoptosis-associated protein Bbk (Accession No. AR043361, U.S. Pat. No. 5,834,234). FIG. 22 shows a comparison of the amino acid sequence between the human protein encoded by clone 55 and the human apoptosis-associated protein Bbk. In this figure, - means a gap, * means the same amino acid residue as the protein of this invention, and . means an amino acid residue similar to the protein of this invention, respectively. This protein in which arginine was inserted between 35th proline and 36th serine in the human apotosis associated protein Bbk lacked 91 amino acid residues from 143rd leucine to 233rd tryptophan of the Bbk.

As a result of referring to GenBank on the basis of the base sequence of cDNA of clone 55, those having a homology of not less than 90% (e.g. Accession No. AA251393) were found to have been registered in EST, but as it is of the partial sequence, it cannot be decided whether or not the same protein as that encoded by clone 55 is encoded.

56: HP10552

As the result of determining the total base sequence of cDNA insert of clone HP10552 obtained from human osteosarcoma cell line Saos-2 cDNA library, it was found that it had a structure of 132 bp 5′ untranslated region, 738 bp ORF and 483 bp 3′ untranslated region (SEQ ID No: 111). The ORF encoded the protein consisting of 245 amino acid residues (SEQ ID No: 112), and as a result of in vitro translation, a translated product of 37 kDa larger than molecular weight 27,609 anticipated from the ORF was produced (Example 2). The fusion protein of this protein and GFP was found to be expressed in the coagulated mass form (Example 4).

As a result of referring to GenBank on the basis of the base sequence of cDNA of clone 56, those having a homology of not less than 90% (e.g. Accession No. AI 929089) were found to have been registered in EST, but as it is of the partial sequence, it cannot be decided whether or not the same protein as that encoded by clone 56 is encoded.

57: HP10553

As the result of determining the total base sequence of cDNA insert of clone HP10553 obtained from human fibrosarcoma cell line HT-1080 cDNA library, it was found that it had a structure of 169 bp 5′ untranslated region, 333 bp ORF and 151 bp 3′ untranslated region (SEQ ID No: 113). The ORF encoded the protein consisting of 110 amino acid residues (SEQ ID No: 114). As a result of in vitro translation, a translated product of 14 kDa larger than molecular weight 12,387 anticipated from ORF was produced (Example 2). The fusion protein of this protein and GFP was expressed in the whole cell (Example 4).

As a result of referring to GenBank on the basis of the base sequence of cDNA of clone 57, those having a homology of not less than 90% (e.g. Accession No. Z43871) were found to have been registered in EST, but as it is of the partial sequence, it cannot be decided whether or not the same protein as that encoded by clone 57 is encoded.

58: HP10558

As the result of determining the total base sequence of cDNA insert of clone HP10558 obtained from human osteosarcoma cell line Saos-2 cDNA library, it was found that it had a structure of 39 bp 5′ untranslated region, 372 bp ORF and 232 bp 3′ untranslated region (SEQ ID No: 115). The ORF encoded the protein consisting of 123 amino acid residues (SEQ ID No: 116). As a result of in vitro translation, a translated product of 20 kDa larger than molecular weight 14,225 anticipated from the ORF (Example 2). The fusion protein of this protein and GFP was localized in the nucleolus (Example 4).

As a result of referring to GenBank on the basis of the base sequence of cDNA of clone 58, those having a homology of not less than 90% (e.g. Accession No. AA327056) were found to have been registered in EST, but as it is of the partial sequence, it cannot be decided whether or not the same protein as that encoded by clone 58 is encoded. Further, a clone (Accession No. AX017850, WO 9946375-A) to coincide with the partial sequence of anti-sense strand was found to have been registered, but it cannot be decided whether or not the same protein as that encoded by clone 58 is encoded by this clone.

59: HP10559

As the result of determining the total base sequence of cDNA insert of clone HP10559 obtained from human osteosarcoma cell line Saos-2 cDNA library, it was found that it had a structure of 305 bp 5′ untranslated region, 714 bp ORF and 274 bp 3′ untranslated region (SEQ ID No: 117). The ORF encoded the protein consisting of 237 amino acid residues (SEQ ID No: 118). The fusion protein of this protein and GFP was localized in the nucleus (Example 4).

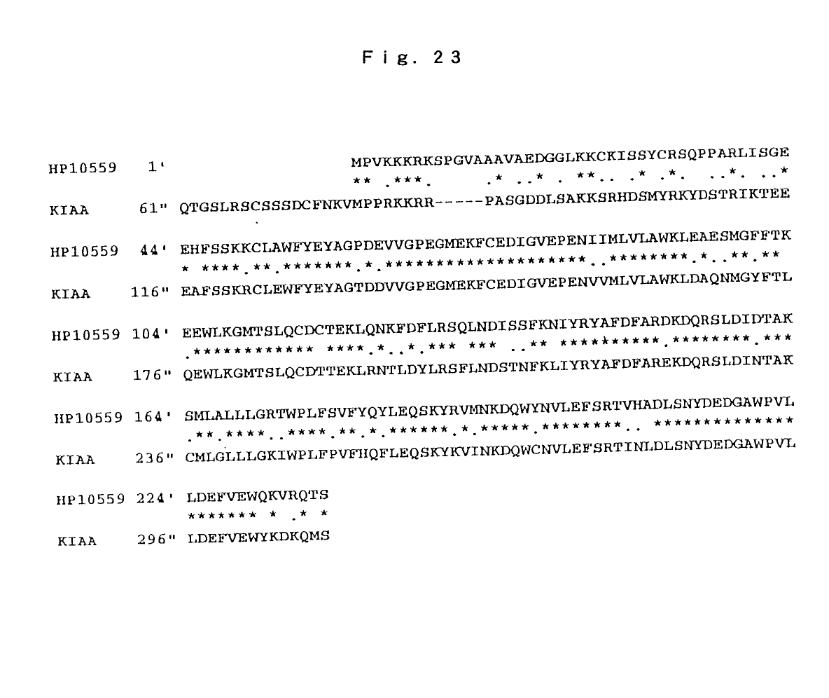

As a result of searching the protein database by using the amino acid sequence of this protein, there was found a similarity to human hypothetical protein KIAA0276 (Accession No. BAA13405). FIG. 23 shows a comparison of the amino acid sequence between the human protein encoded by clone 59 and the human hypothetical protein KIAA0276. In this figure, - means a gap, * means the same amino acid residue as the protein of this invention, and . means an amino acid residue similar to the protein of this invention, respectively. Over the whole region, they had a homology of 69.6%.

As a result of referring to GenBank on the basis of the base sequence of cDNA of clone 59, those having a homology of not less than 90% (e.g. Accession No. A75334, WO 9401548) and other having a homology of not less than 90% (e.g. Accession No. AA099966) were found to have been registered in EST, but as they are of the partial sequence, it cannot be decided whether or not the same protein as that encoded by clone 59 is encoded by this clone.

60: HP10560

As the result of determining the total base sequence of cDNA insert of clone HP10560 obtained from human osteosarcoma cell line Saos-2 cDNA library, it was found that it had a structure of 147 bp 5′ untranslated region, 324 bp ORF and 445 bp 3′ untranslated region (SEQ ID No: 119). The ORF encoded the protein consisting of 107 amino acid residues (SEQ ID No: 120). The fusion protein of this protein and GFP was expressed in the whole cell (Example 4).

As a result of referring to GenBank on the basis of the base sequence of cDNA of clone 60, those having a homology of not less than 90% (e.g. Accession No. C17870) were found to have been registered in EST, but as it is of the partial sequence, it cannot be decided whether or not the same protein as that encoded by clone 60 is encoded.

61: HP10561

As the result of determining the total base sequence of cDNA insert of clone HP10561 obtained from human stomach carcinoma cDNA library, it was found that it had a structure of 50 bp 5′ untranslated region, 681 bp ORF and 271 bp 3′ untranslated region (SEQ ID No: 121). The ORF encoded the protein consisting of 226 amino acid residues (SEQ ID No: 122). As a result of in vitro translation, a translated product of 29 kDa larger than molecular weight 22,581 anticipated from the ORF (Example 2). The fusion protein of this protein and GFP was localized in the nucleolus (Example 4).

As a result of referring to GenBank on the basis of the base sequence of cDNA of clone 61, those having a homology of not less than 90% (e.g. Accession No. W84353) were found to have been registered in EST, but as it is of the partial sequence, it cannot be decided whether or not the same protein as that encoded by clone 61 is encoded.

62: HP10562

As the result of determining the total base sequence of cDNA insert of clone HP10562 obtained from human osteosarcoma cell line Saos-2 cDNA library, it -was found that it had a structure of 267 bp 5′ untranslated region, 1188 bp ORF and 298 bp 3′ untranslated region (SEQ ID No: 123). The ORF encoded the protein consisting of 395 amino acid residues (SEQ ID No: 124). As a result of in vitro translation, a translated product of 48 kDa larger than molecular weight 43,405 anticipated from ORF (Example 2). The fusion protein between this protein and GFP was expressed in the granular form or weakly expressed in the whole cell (Example 4).

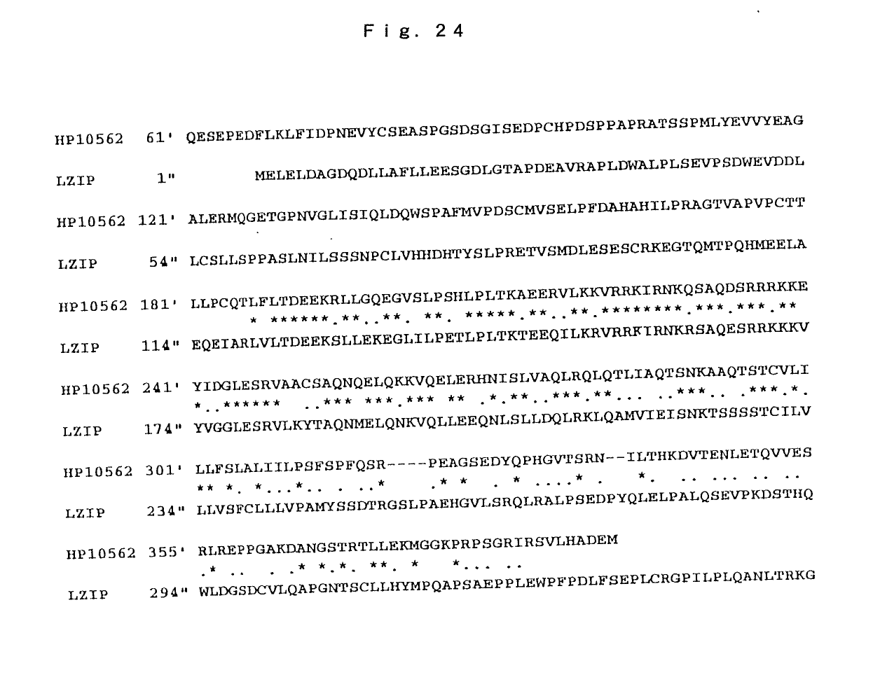

As a result of referring to the protein database on the basis of the amino acid sequence of this protein, there was found a similarity to human basic leucine zipper protein LZIP (Accession No. BAA13405). FIG. 24 shows a comparison of the amino acid sequence between human protein encoded by clone 62 and human basic leucine zipper protein LZIP. In this figure, - means a gap, * means the same amino acid residue as the protein of this invention, and . means an amino acid residue similar to the protein of this invention, respectively. In the 206 amino acid residues of the intermediary region, they had a homology of 43.7%.

As a result of referring to GenBank on the basis of the base sequence of cDNA of clone 62, those having a homology of not less than 90% (e.g. Accession No. AA203110) were found to have been registered in EST, but as it is of the partial sequence, it cannot be decided whether or not the same protein as that encoded by clone 62 is encoded.

63: HP10564

As the result of determining the total base sequence of cDNA insert of clone HP10564 obtained from human osteosarcoma cell line Saos-2 cDNA library, a structure of 53 bp 5′ untranslated region, 69 bp ORF and 546 bp 3′ untranslated region (SEQ ID No: 125). The ORF encoded the protein consisting of 22 amino acid residues (SEQ ID No: 126). The fusion protein of this protein and GFP was expressed in the whole cell (Example 4).

As a result of referring to GenBank on the basis of the base sequence of cDNA of clone 63, those having a homology of not less than 90% (e.g. Accession No. AI 879105) were found to have been registered in EST, but as it is of the partial sequence, it cannot be decided whether or not the same protein as that encoded by clone 63 is encoded.

64: HP10569

As the result of determining the total base sequence of cDNA insert of clone HP10569 obtained from human epidermal carcinoma cell line KB cDNA library, a structure of 26 bp 5′ untranslated region, 213 bp ORF and 40 bp 3′ untranslated region (SEQ ID No: 127). The ORF encoded the protein consisting of 70 amino acid residues (SEQ ID No: 128). As a result of in vitro translation, a translated product of 9 kDa almost same as molecular weight 8,691 anticipated from the ORF (Example 2). The fusion protein of this protein and GFP was expressed in the whole cell (Example 4).

As a result of referring to GenBank on the basis of the base sequence of cDNA of clone 64, those having a homology of not less than 90% (e.g. Accession No. AI 376841) were found to have been registered in EST, but as it is of the partial sequence, it cannot be decided whether or not the same protein as that encoded by clone 64 is encoded.

65: HP10601

As the result of determining the total base sequence of cDNA insert of clone HP10601 obtained from human fibrosarcoma cell line HT-1080 cDNA library, it was found that it had a structure of 90 bp 5′ untranslated region, 2088 bp ORF and 1189 bp 3′ untranslated region (SEQ ID No: 129). The ORF encoded the protein consisting of 695 amino acid residues (SEQ ID No: 130). As a result of in vitro translation, a translated product of 81 kDa larger than molecular weight 76, 105 anticipated from the ORF is produced (Example 2). The fusion protein of this protein and GFP was expressed in the nucleus or in the particle form (Example 4).

As a result of referring to GenBank on the basis of the base sequence of cDNA of clone 65, those having a homology of not less than 90% (e.g. Accession No. R97122) were found to have been registered in EST, but as it is of the partial sequence, it cannot be decided whether or not the same protein as that encoded by clone 65 is encoded.

66: HP10456

As the result of determining the total base sequence of cDNA insert of clone HP10456 obtained from human osteosarcoma cell line U-2 cDNA library, it was found that it had a structure of 99 bp 5′ untranslated region, 600 bp ORF and 591 bp 3′ untranslated region (SEQ ID No: 131). The ORF encoded the protein consisting of 199 amino acid residues (SEQ ID No: 132). As a result of in vitro translation, a translated product of 31 kDa larger than molecular weight 22,095 anticipated from the ORF was produced (Example 2). The fusion protein of this protein and GFP was expressed in the cytoplasm as a coagulated mass (Example 4).

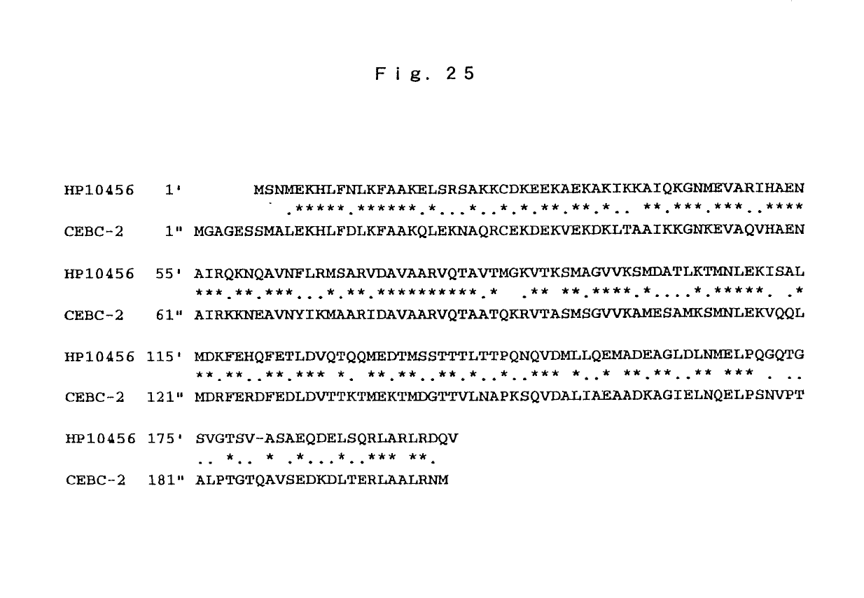

As a result of searching the protein database by using the amino acid sequence of this protein, there was found a similarity to nematoda BC-2-like protein LZIP (Accession No. AAD03134). FIG. 25 shows a comparison of the amino acid sequence between the human protein encoded by clone 66 and the nematoda BC-2-like protein. In this figure, - means a gap, * means the same amino acid residue as the protein of this invention, and . means an amino acid residue similar to the protein of this invention, respectively. Over the whole region, they had a homology of 56.9%.

As a result of referring to GenBank on the basis of the base sequence of cDNA of clone 66, those having a homology of not less than 90% (e.g. Accession No. C04706) were found to have been registered in EST, but as it is of the partial sequence, it cannot be decided whether or not the same protein as that encoded by clone 66 is encoded.

67: HP10498

As the result of determining the total base sequence of cDNA insert of clone HP10498 obtained from human osteosarcoma cell line Saos-2 cDNA library, it was found that it had a structure of 23 bp 5′ untranslated region, 357 bp ORF and 184 bp 3′ untranslated region (SEQ ID No: 133). The ORF encoded the protein consisting of 118 amino acid residues (SEQ ID No: 134). As a result of in vitro translation, a translated product of 14 Da almost same as molecular weight 13,466 anticipated from the ORF was produced (Example 2). The fusion protein of this protein and GFP was expressed in the whole cell (Example 4).

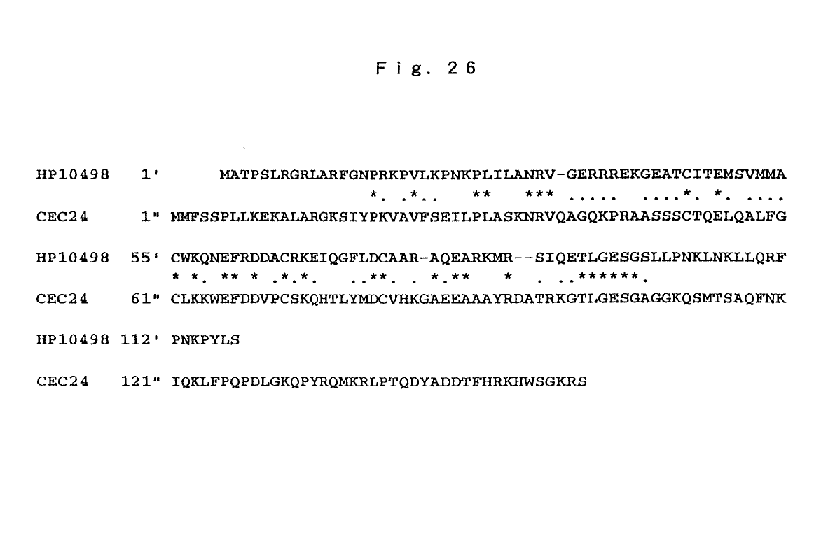

As a result of searching the protein database by using the amino acid sequence of this protein, there was found a similarity to nematoda hypothetical protein C24D19.6 (Accession No. AAB88317). FIG. 26 shows a comparison of the amino acid sequence between the human protein encoded by clone 67 and the nematoda hypothetical protein C24D19.6. In this figure, - means a gap, * means the same amino acid residue as the protein of this invention, and . means an amino acid residue similar to the protein of this invention, respectively. In the 87 amino acid residues of the intermediary region, they had a homology of 32.2%.

As a result of referring to GenBank on the basis of the base sequence of cDNA of clone 67, those having a homology of not less than 90% (e.g. Accession No. AA431880) were found to have been registered in EST, but as it is of the partial sequence, it cannot be decided whether or not the same protein as that encoded by clone 67 is encoded.

68: HP10503

As the result of determining the total base sequence of cDNA insert of clone HP10503 obtained from human osteosarcoma cell line Saos-2 cDNA library, it was found that it had a structure of 466 bp 5′ untranslated region, 345 bp ORF and 93 bp 3′ untranslated region (SEQ ID No: 135). The ORF encoded the protein consisting of 114 amino acid residues (SEQ ID No: 136). The fusion protein of this protein and GFP was expressed in the whole cell (Example 4).

As a result of referring to GenBank on the basis of the base sequence of cDNA of clone 68, those having a homology of not less than 90% (e.g. Accession No. AA 305157) were found to have been registered in EST, but as it is of the partial sequence, it cannot be decided whether or not the same protein as that encoded by clone 68 is encoded.

69: HP10505

As the result of determining the total base sequence of cDNA insert of clone HP10505 obtained from human osteosarcoma cell line Saos-2 cDNA library, it was found that it had a structure of 89 bp 5′ untranslated region, 264 bp ORF and 119 bp 3′ untranslated region (SEQ ID No: 137). The ORF encoded the protein consisting of 87 amino acid residues (SEQ ID No: 138). As a result of in vitro translation, a translated product of 14 Da larger than molecular weight 10,734 anticipated from the ORF was produced (Example 2). The fusion protein of this protein and GFP was localized in the mitochondria (Example 4).

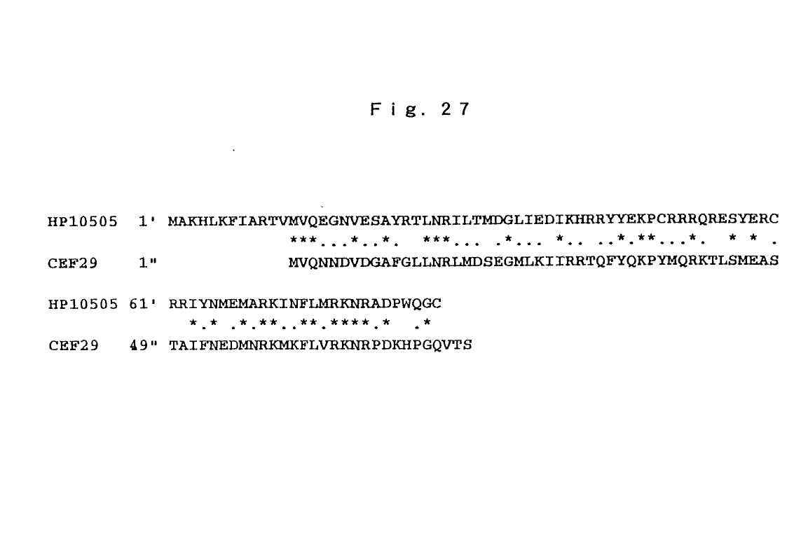

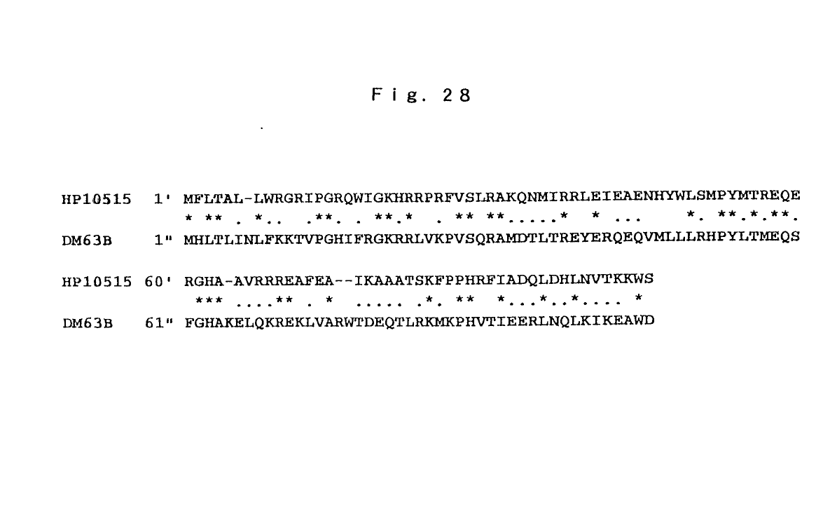

As a result of searching the protein database by suing the amino acid sequence of this protein, there was found a similarity to nematoda hypothetical protein F29B9.10 (Accession No. AAB09120). The nematoda hypothetical protein F29B9.10 has 30S ribosome and a weak similarity. FIG. 27 shows a comparison of the amino acid sequence between the human protein encoded by clone 69 and the nematoda hypothetical protein F29B9.10. In this figure, - means a gap, * means the same amino acid residue as the protein of this invention, and . means an amino acid residue similar to the protein of this invention, respectively. Over the 74 amino acid residues except for the N-terminal, they had a homology of 39.2%.

As a result of referring to GenBank on the basis of the base sequence of cDNA of clone 69, those having a homology of not less than 90% (e.g. Accession No. AA029070) were found to have been registered in EST, but as it is of the partial sequence, it cannot be decided whether or not the same protein as that encoded by clone 69 is encoded.

70: HP10511

As the result of determining the total base sequence of cDNA insert of clone HP10511 obtained from human stomach carcinoma cDNA library, it was found that it had a structure of 48 bp 5′ untranslated region, 120 bp ORF and 12 bp 3′ untranslated region (SEQ ID No: 139). The ORF encoded the protein consisting of 39 amino acid residues (SEQ ID No: 140). As a result of in vitro translation, a translated product of 4 kDa almost same as molecular weight 3,939 anticipated from the ORF (Example 2). The fusion protein of this protein and GFP was expressed in the whole cell (Example 4).

As a result of referring to GenBank on the basis of the base sequence of cDNA of clone 70, those having a homology of not less than 90% (e.g. Accession No. AA629178) were found to have been registered in EST, but as it is of the partial sequence, it cannot be decided whether or not the same protein as that encoded by clone 70 is encoded.

71: HP10515

As the result of determining the total base sequence of cDNA insert of clone HP10515 obtained from human liver cDNA library, it was found that it had a structure of 34 bp 5′ untranslated region, 309 bp ORF and 130 bp 3′ untranslated region (SEQ ID No: 141). The ORF encoded the protein consisting of 102 amino acid residues (SEQ ID No: 142). As a result of in vitro translation, a translated product of 15Da larger than molecular weight 12,259 anticipated from the ORF was produced (Example 2). The fusion protein of this protein and GFP was expressed in the cytoplasm as the particle form (Example 4).

As a result of searching the protein database by using the amino acid sequence of this protein, there was found a similarity to drosophila hypothetical protein 63B12.s (Accession No. CAA15941). FIG. 28 shows a comparison of the amino acid sequence between the human protein encoded by clone 71 and the drosophila hypothetical protein 63B12.s. In this figure, - means a gap, * means the same amino acid residue as the protein of this invention, and . means an amino acid residue similar to the protein of this invention, respectively. Over the whole region, they had a homology of 32.4%.

As a result of referring to GenBank on the basis of the base sequence of cDNA of clone 71, those having a homology of not less than 90% (e.g. Accession No. AA349062) were found to have been registered in EST, but as it is of the partial sequence, it cannot be decided whether or not the same protein as that encoded by clone 71 is encoded.

72: HP01124

As the result of determining the total base sequence of cDNA insert of clone HP01124 obtained from human liver cDNA library, it was found that it had a structure of 105 bp 5′ untranslated region, 1026 bp ORF and 533 bp 3′ untranslated region (SEQ ID No: 143). The ORF encoded the protein consisting of 341 amino acid residues (SEQ ID No: 144). As a result of in vitro translation, a translated product of 37 kDa almost same as molecular weight 37,786 anticipated from the ORF was produced (Example 2). The fusion protein of this protein and GFP was localized in the nucleus (Example 4).

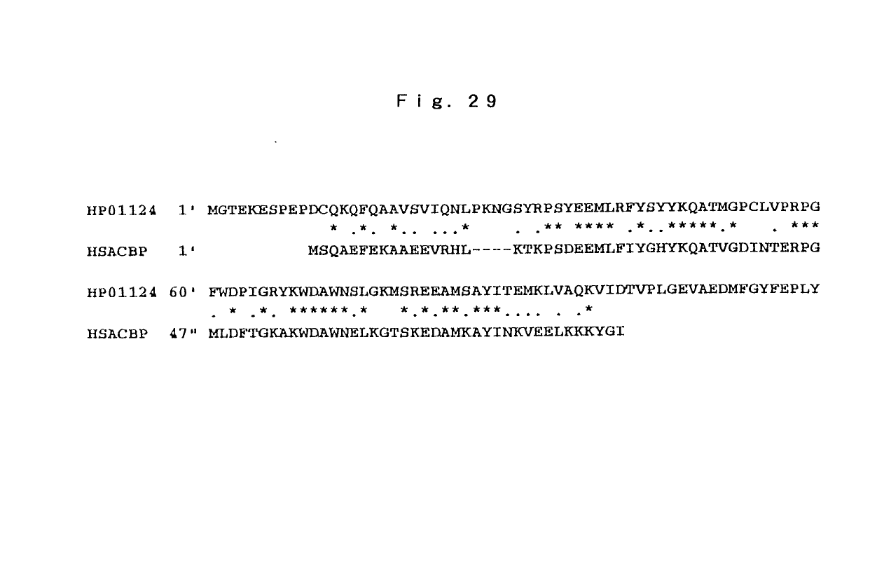

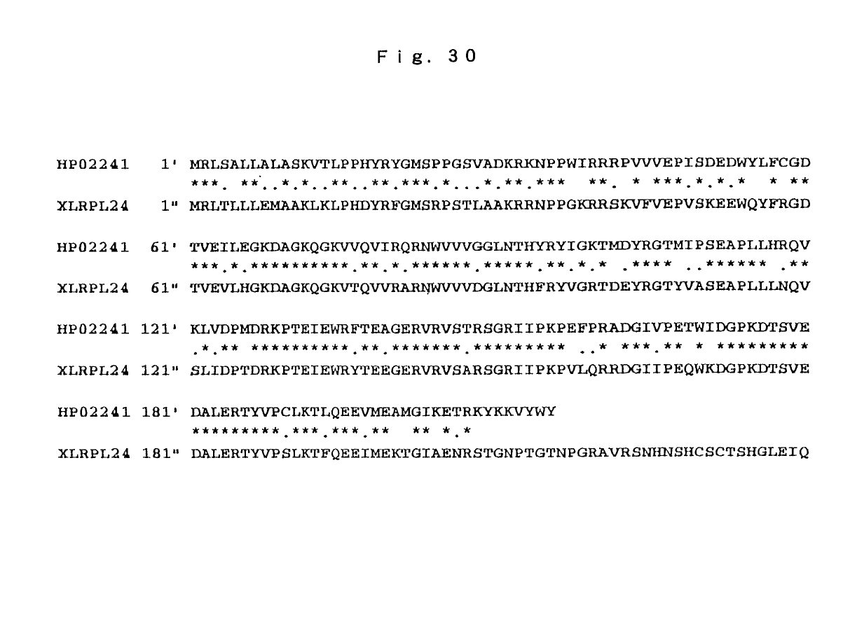

As a result of searching the protein database by using the amino acid sequence of this protein, there was found a similarity to human Acyl-CoA-binding protein (Accession No. P07108). FIG. 29 shows a comparison of the amino acid sequence between the human protein encoded by clone 72 and the human Acyl-CoA-binding protein. In this figure, - means a gap, * means the same amino acid residue as the protein of this invention, and . means an amino acid residue similar to the protein of this invention, respectively. Over the whole region, they had a homology of 43.0%.

As a result of referring to GenBank on the basis of the base sequence of cDNA of clone 72, those having a homology of not less than 90% (e.g. Accession No. N41542) were found to have been registered in EST, but as it is of the partial sequence, it cannot be decided whether or not the same protein as that encoded by clone 72 is encoded.

73: HP02241

As the result of determining the total base sequence of cDNA insert of clone HP02241 obtained from human stomach carcinoma cDNA library, it was found that it had a structure of 89 bp 5′ untranslated region, 651 bp ORF and 95 bp 3′ untranslated region (SEQ ID No: 145). The ORF encoded the protein consisting of 216 amino acid residues (SEQ ID No: 146). As a result of in vitro translation, a translated product of 30 kDa larger than molecular weight 24,899 anticipated from the ORF (Example 2). The fusion protein of this protein and GFP was expressed as a partially coagulated mass in the whole cell (Example 4).

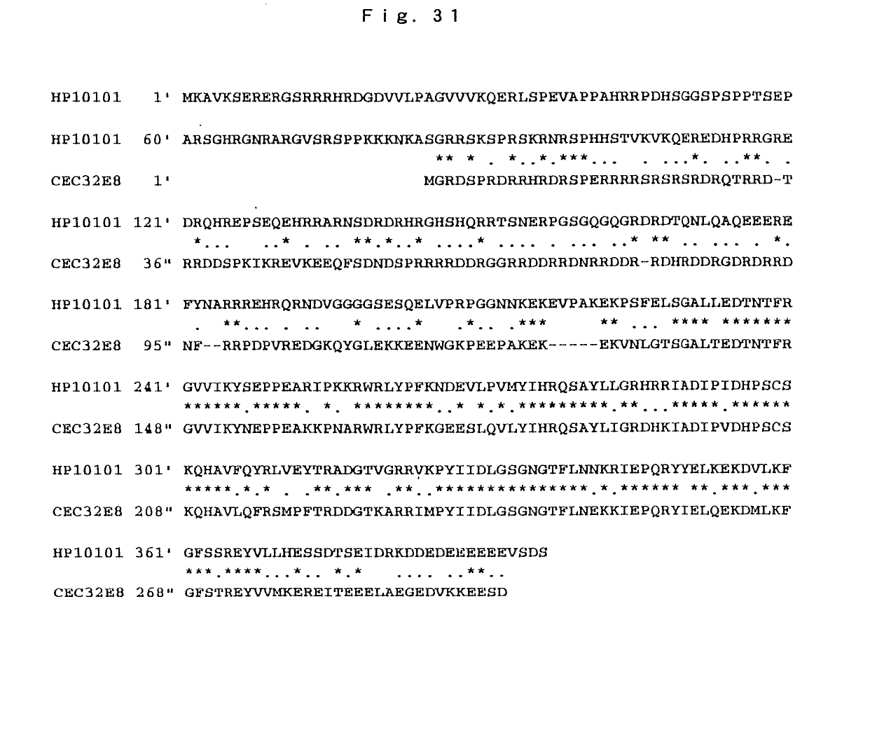

As a result of searching the protein database by using the amino acid sequence of this protein, there was found a similarity to Xenopus ribosomal protein L24-like protein (Accession No. CAB40554). FIG. 30 shows a comparison of the amino acid sequence between the human protein encoded by clone 73 and the Xenopus ribosomal protein L24-like protein. In this figure, - means a gap, * means the same amino acid residue as the protein of this invention, and . means an amino acid residue similar to the protein of this invention, respectively. Over the 208 amino acid residues of the N-terminal, they had a homology of 69.7%.

As a result of referring to GenBank on the basis of the base sequence of cDNA of clone 73, those having a homology of not less than 90% (e.g. Accession No. AL 038493) were found to have been registered in EST, but as they are of the partial sequence, it cannot be decided whether or not the same protein as that encoded by clone 73 is encoded by this clone.

74: HP10101

As the result of determining the total base sequence of cDNA insert of clone HP10101 obtained from human fibrosarcoma cell line HT-1080 cDNA library, it was found that it had a structure of 70 bp 5′ untranslated region, 119 1 bp ORF and 1204 bp 3′ untranslated region (SEQ ID No: 147). The ORF encoded the protein consisting of 396 amino acid residues (SEQ ID No: 148). As a result of in vitro translation, a translated product of 54 kDa larger than molecular weight 45,750 anticipated from the ORF (Example 2). The fusion protein of this protein and GFP was expressed in the granular form in the nucleus (Example 4).

As a result of searching the protein database by using the amino acid sequence of this protein, there was found a similarity to nematoda hypothetical protein C32E8.5 (Accession No. AAB 42323). FIG. 31 shows a comparison of the amino acid sequence between the human protein encoded by clone 74 and the nematoda hypothetical protein C32E8.5. In this figure, - means a gap, * means the same amino acid residue as the protein of this invention, and . means an amino acid residue similar to the protein of this invention, respectively. Over the 307 amino acid residues in the C-terminal, they had a homology of 46.9%.

As a result of referring to GenBank on the basis of the base sequence of cDNA of clone 74, those having a homology of not less than 90% (e.g. Accession No. AA 460870) were found to have been registered in EST, but as they are of the partial sequence, it cannot be decided whether or not the same protein as that encoded by clone 74 is encoded by this clone.

75: HP10370

As the result of determining the total base sequence of cDNA insert of clone HP10370 obtained from human epidermal carcinoma cell line KB cDNA library, it was found that it had a structure of 148 bp 5′ untranslated region, 1356 bp ORF and 2096 bp 3′ untranslated region (SEQ ID No: 149). The ORF encoded the protein consisting of 451 amino acid residues (SEQ ID No: 150). The fusion protein of this protein and GFP was expressed in the whole cell (Example 4).

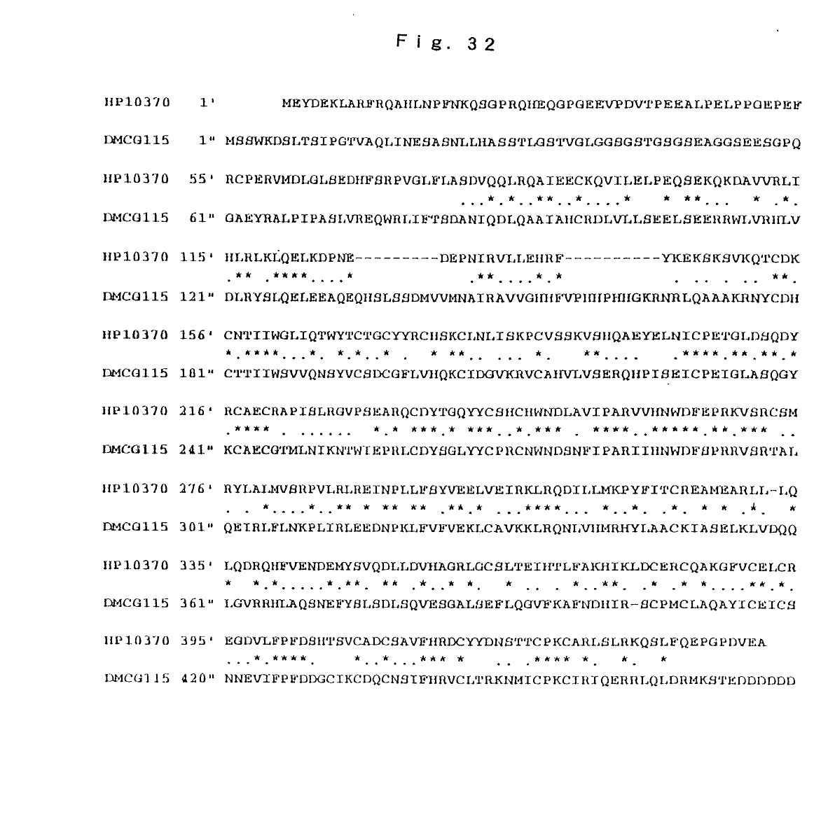

As a result of searching the protein database by using the amino acid sequence of this protein, there was found a similarity to drosophila hypothetical protein CG11534 (Accession No. AAF49957). FIG. 32 shows a comparison of the amino acid sequence between the human protein encoded by clone 75 and the drosophila hypothetical protein CG11534. In this figure, - means a gap, * means the same amino acid residue as the protein of this invention, and . means an amino acid residue similar to the protein of this invention, respectively. Over the 382 amino acid residues in the C-terminal, they had a homology of 36.9%.

As a result of referring to GenBank on the basis of the base sequence of cDNA of clone 75, those having a homology of not less than 90% (e.g. Accession No. AA035322) were found to have been registered in EST, but as they are of the partial sequence, it cannot be decided whether or not the same protein as that encoded by clone 75 is encoded by this clone.

76: HP10427

As the result of determining the total base sequence of cDNA insert of clone HP10427 obtained from human stomach carcinoma cDNA library, it was found that it had a structure of 11 bp 5′ untranslated region, 342 bp ORF and 89 bp 3′ untranslated region (SEQ ID No: 151). The ORF encoded the protein consisting of 113 amino acid residues (SEQ ID No: 152). The fusion protein of this protein and GFP was localized in the Golgi body (Example 4).

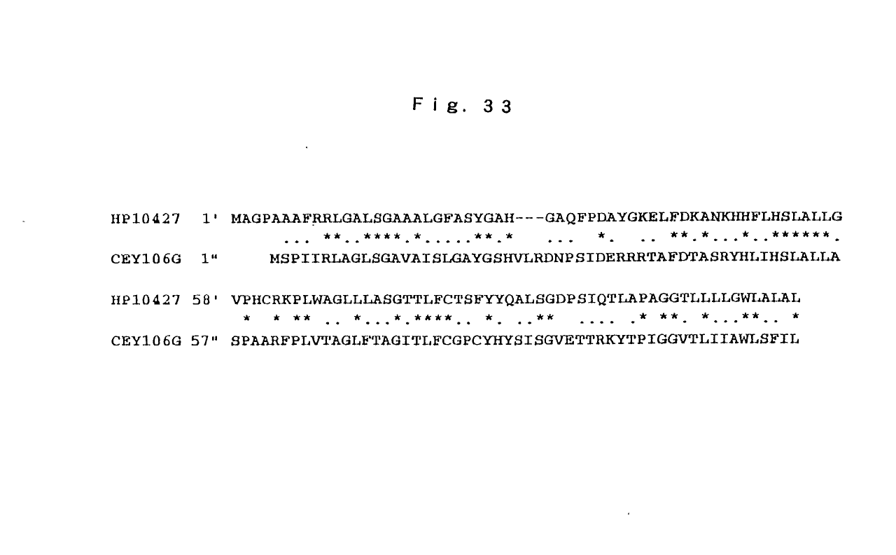

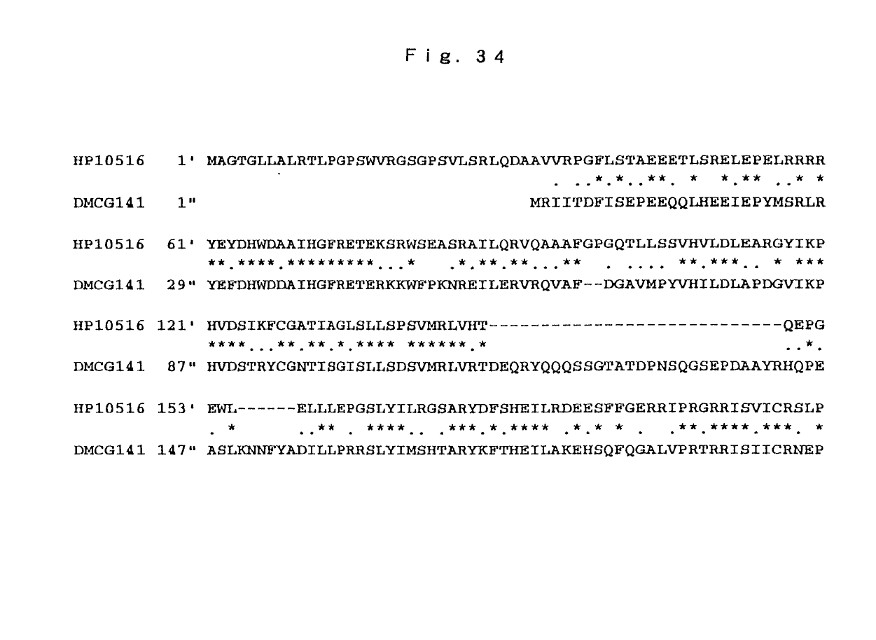

As a result of searching the protein database by using the amino acid sequence of this protein, there was found a similarity to nematoda hypothetical protein Y106G6H. 8 (Accession No. CAB 6338). FIG. 33 shows a comparison of the amino acid sequence between the human protein encoded by clone 76 and the nematoda hypothetical protein Y106G6H. 8. In this figure, - means a gap, * means the same amino acid residue as the protein of this invention, and . means an amino acid residue similar to the protein of this invention, respectively. Over the whole region, they had a homology of 36.9%.

As a result of referring to GenBank on the basis of the base sequence of cDNA of clone 76, those having a homology of not less than 90% (e.g. Accession No. R76178) were found to have been registered in EST, but as they are of the partial sequence, it cannot be decided whether or not the same protein as that encoded by clone 76 is encoded by this clone.

77: HP10438

As the result of determining the total base sequence of cDNA insert of clone HP10438 obtained from human stomach carcinoma 2 cDNA library, it was found that it had a structure of 11 bp 5′ untranslated region, 669 bp ORF and 46 bp 3′ untranslated region (SEQ ID No: 153). The ORF encoded the protein consisting of 222 amino acid residues (SEQ ID No: 154). As a result of in vitro translation, a translated product of 28 kDa slightly larger than molecular weight 25,384 anticipated from the ORF was produced (Example 2). The fusion protein of this protein and GFP was expressed in the nucleus (Example 4).

As a result of referring to GenBank on the basis of the base sequence of cDNA of clone 77, those having a homology of not less than 90% (e.g. Accession No. AA088470) were found to have been registered in EST, but as they are of the partial sequence, it cannot be decided whether or not the same protein as that encoded by clone 77 is encoded.

78: HP10502

As the result of determining the total base sequence of cDNA insert of clone HP10502 obtained from human fibrosarcoma cell line HT-1080 cDNA library, it was found that it had a structure of 207 bp 5′ untranslated region, 837 bp ORF and 76 bp 3′ untranslated region (SEQ ID No:. 155). The ORF encoded the protein consisting of 278 amino acid residues (SEQ ID No: 156). The fusion protein of this protein and GFP was expressed in the nucleus (Example 4).

As a result of referring to GenBank on the basis of the base sequence of cDNA of clone 78, those having a homology of not less than 90% (e.g. Accession No. AA648423) were found to have been registered in EST, but as they are of the partial sequence, it cannot be decided whether or not the same protein as that encoded by clone 78 is encoded by this clone.

79: HP10516

As the result of determining the total base sequence of cDNA insert of clone HP10516 obtained from human stomach carcinoma cDNA library, it was found that it had a structure of 26 bp 5′ untranslated region, 666 bp ORF and 55 bp 3′ untranslated region (SEQ ID No:. 157). The ORF encoded the protein consisting of 221 amino acid residues (SEQ ID No: 158). The fusion protein of this protein and GFP was expressed in the whole cell (Example 4).

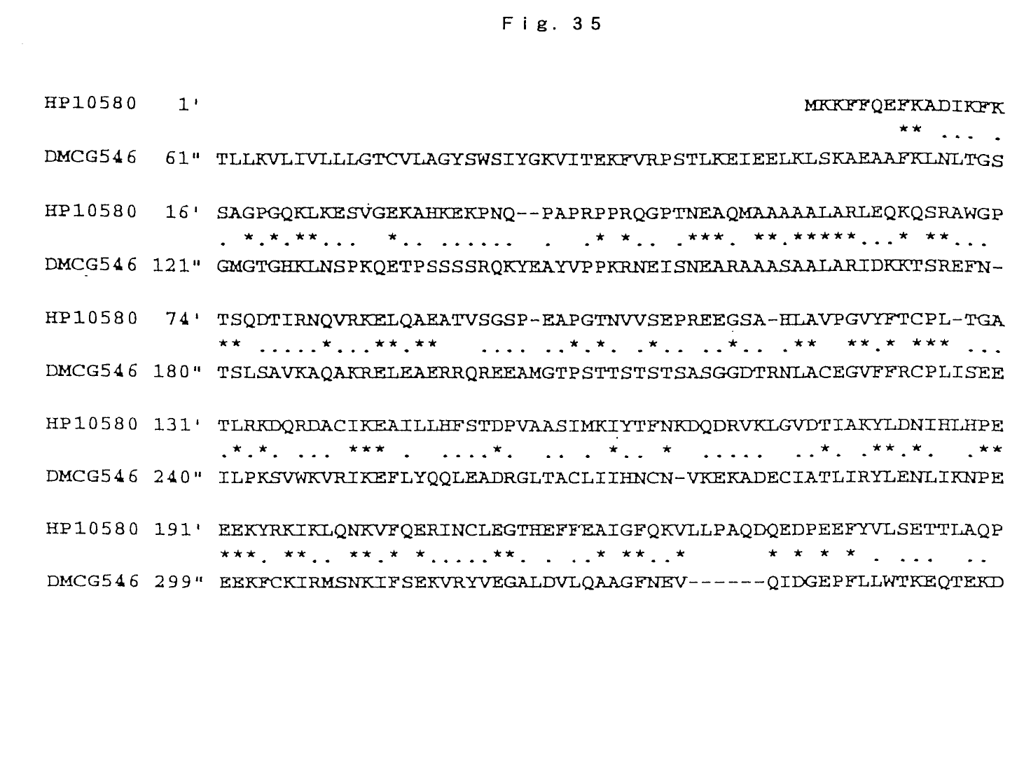

As a result of searching the protein database by using the amino acid sequence of this protein, there was found a similarity to drosophila hypothetical protein CG14130 (Accession No. AAF50005). FIG. 34 shows a comparison of the amino acid sequence between the human protein encoded by clone 79 and the drosophila hypothetical protein CG14130. In this figure, - means a gap, * means the same amino acid residue as the protein of this invention, and . means an amino acid residue similar to the protein of this invention, respectively. Over the whole region, they had a homology of 36.9%.

As a result of referring to GenBank on the basis of the base sequence of cDNA of clone 79, those having a homology of not less than 90% (e.g. Accession No. AW 245556) were found to have been registered in EST, but as they are of the partial sequence, it cannot be decided whether or not the same protein as that encoded by clone 79 is encoded by this clone.

80: HP10580

As the result of determining the total base sequence of cDNA insert of clone HP10580 obtained from human stomach carcinoma cDNA library, it was found that it had a structure of 94 bp 5′ untranslated region, 1326 bp ORF and 21 bp 3′ untranslated region (SEQ ID No:. 159). The ORF encoded the protein consisting of 441 amino acid residues (SEQ ID No: 160). The fusion protein of this protein and GFP was expressed as a coagulated mass in the cytoplasm (Example 4).

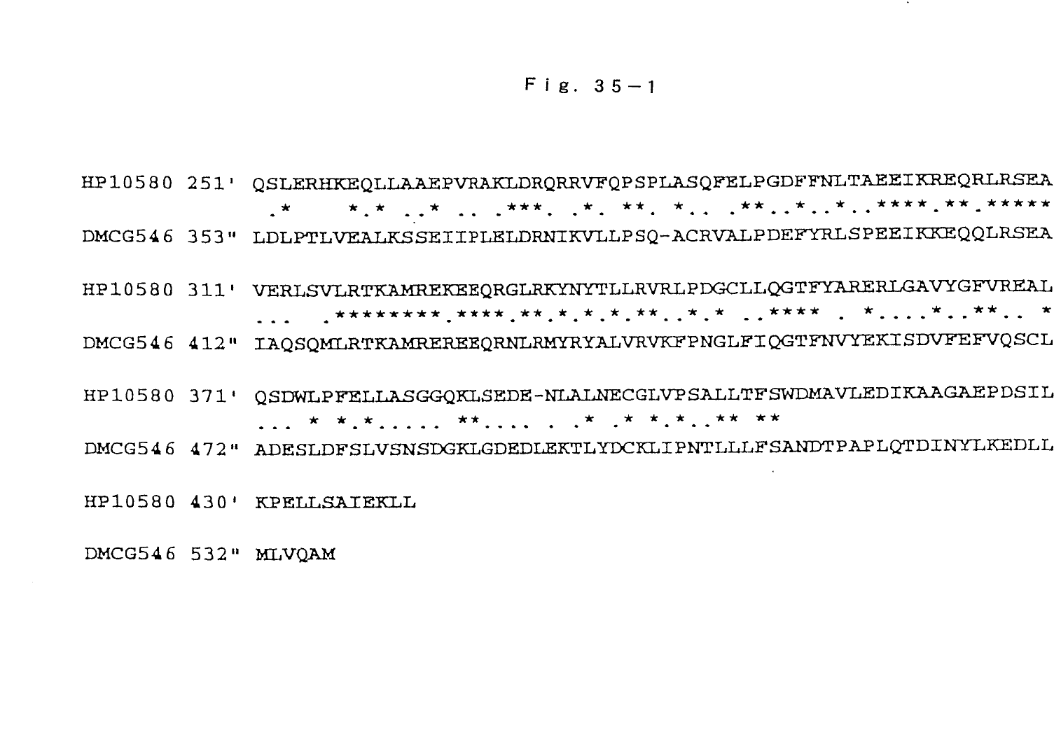

As a result of searching the protein database by using the amino acid sequence of this protein, there was found a similarity to drosophila hypothetical protein CG5469 (Accession No. AAF50005). FIG. 35 shows a comparison of the amino acid sequence between the human protein encoded by clone 80 and the drosophila hypothetical protein CG5469. In this figure, - means a gap, * means the same amino acid residue as the protein of this invention, and . means an amino acid residue similar to the protein of this invention, respectively. Over the whole region, they had a homology of 35.0%.

As a result of referring to GenBank on the basis of the base sequence of cDNA of clone 80, those having a homology of not less than 90% (e.g. Accession No. AI 188741) were found to have been registered in EST, but as they are of the partial sequence, it cannot be decided whether or not the same protein as that encoded by clone 80 is encoded by this clone.

### Example 2

### Protein Synthesis by in vitro Translation

Using plasmid vector having cDNA isolated in Example 1, in vitro transcription/translation due to T_{N}T rabbit reticulocyte lysate kit (Promega Co.) was performed. In this case, the expressed product was labelled with a radioisotope, using [³⁵S]methionine. Each reaction was carried out according to the protocol attached to the kit.

Practical procedure was as follows: 2 μg of plasmid was allowed to react in a reaction mixture (total amount: 252 μl) containing 12.5 μl of T_{N}T rabbit reticulocyte lysate, 2 μl (0.37 MBq/μl) of [³⁵S]methionine (Amersham Co.), 0.5 μl of T7RNA polymerase and 20 U of RNasin at 30° C. for 90 minutes. The reaction mixture (3 μl) was mixed with 2 μl of SDS sampling buffer (125 mM Tris-HCl buffer, pH 6.8, 120 mM 2-mercaptoethanol, 2% SDS solution, 0.025% Bromophenol Blue, 20% glycerol), heated at 95° C. for 3 minutes and subjected to electrophoresis with SDS-polyacrylamide gel. Molecular weight of the translated product was obtained by performing the autoradiography.

### Example 3

### Expression in COS7 cells

E. coli transformed by expression vector containing cDNA isolated in Example 1 was cultivated at 37° C. for 2 hours in 2 ml of 2xYT medium containing 100 μg/ml ampicillin, and the mixture was mixed with helper phage M13KO7 (50 μl) and cultivated at 37° C. overnight. The culture broth was centrifuged, and the separated supernatant was treated with polyethylene glycol. The precipitated single-stranded phage particle was suspended in 100 μl of 1 mM Tris-0.1 mM EDTA (pH 8 (TE)).

Culture cell COS7 derived from monkey kidney was cultivated at 37° C. in the presence of 5% CO₂ in Dulbecco's modified Eagle medium (DMEM) containing 10% fetal bovine serum. COS7 cells (1×10⁵) were inoculated onto 6-well plate (well diameter, 3 cm; Nunk Co.) and cultivated at 37° C. for 22 hours in the presence of 5% CO₂. After removing the medium, the cell surface was washed with phosphate buffer and then with DMEM containing 50 mM Tris-HCl (pH 7.5) (TDMEM). To this cell were added 1 μl of a suspension of single-stranded phage, 0.6 ml of DMEM medium and 3 μl of TRANSFECTAM™ (IBF Co.), and the resultant suspension was cultivated at 37° C. for 3 hours in the presence of 5% CO₂. The sample liquid is removed, and the cell surface was washed with TDMEM, mixed with 2 ml/well of DMEM containing 10% fetal bovine serum and cultivated at 37° C. for 2 days in the presence of 5% CO₂. The medium was replaced by a medium containing [³⁵S]cysteine or [³⁵S]methionine and cultivated for 1 hour. The broth was centrifuged to separate the medium from the cell, and the protein in the cell fraction was subjected to SDS-PAGE.

### Example 4

### Expression of Green Fluorescent Protein (GFP) Fusion Protein

Using 26 mer of sense primer starting from the translation initiating codon containing the recognition site EcoRI and 26 mer of anti-sense primer inclusive of up to the stop codon containing the recognition site BamHI, the translation region was amplified due to PCR by using as a template cDNA encoding the objective protein. The PCR product was digested with EcoRI and BamHI and incorporated into EcoRI-BamHI site of the vector pEGFP-N1 (manufactured by Clontech Co.) for expressing GFP fusion protein. After confirming the base sequence, the resultant fusion gene expression vector was transfected into COS7 cells according to the method described in Example 3. The region wherein the objective protein was localized was examined by observing the green fluorescent distribution by a fluorescent microscope.

### Example 5

### Preparation of Antibody

Using 26 mer of sense primer starting from the translation initiating codon containing the recognition site EcoRI and 26 mer of anti-sense primer inclusive of up to the stop codon containing the recognition sequence SalI, the translated region was amplified due to PCR by using as a template each cDNA. The PCR product was digested with EcoRI and SalI and incorporated into the EcoRI-SalI site of the pGEX-5X-1 (manufactured: Pharmacia Co.). After confirming the base sequence, the host E. coli JM109 was transfected. The cell was cultivated at 37° C. for 5 hours in LB medium, added with IPTG so as to 0.4 mM of the final concentration and cultivated at 37° C. for 4 hours. The cell was separated by centrifuging, dissolved in a lytic solution (50 mM Tris-HCl pH 7.5, 1 mM EDTA, 0.2 mM PMF), once frozen at −80° C., thawed, and disrupted by sonication. The resultant lysate was centrifuged at 10,000×g for 30 minutes, and the supernatant was added with glutathione sephalose 4B and incubated at 4° C. for 1 hour. The beads were sufficiently washed, and the fusion protein was eluted with an eluent (50 mM Tris-HCl pH 7.5, 50 mM glutathione). A rabbit was immunized with the resultant fusion protein as an antigen in a conventional manner to give an antiserum. The antiserum was purified by subjecting the 40% saturated ammonium sulfate-precipitated fraction to GST affinity column for removing GST antibody. The eluted fraction was further purified by using an antigen column of GST fusion protein.

### INDUSTRIAL APPLICABILITY

As described in detail above, the present application is to provide a purified human protein, DNA fragment encoding the protein, expression vector for the DNA fragment, various cells transfected with the expression vector, and antibody against the protein. The protein provided by this application is useful for detecting the corresponding receptor or ligand as an intracellular targeting protein, for screening novel small molecule medicinals and so on, since each protein is considered to function within a cell. Further, the protein is useful as an antigen for manufacturing the antibody against the proteins. The DNA fragment provided by this application is useful as a probe for gene diagnosis or as a gene source for gene therapy. Further, the DNA fragment can be also used as a gene source for mass production of the protein. The cells in which the protein has been expressed by incorporating the gene can be used for preparing modified forms of the protein. The antibody provided by the present application can be used for detection, determination, purification or the like of the protein.

1. A purified human proteins having any one of the amino acid sequences of SEQ ID No: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158 or 160.

2. A DNA fragment encoding the protein of claim 1.

3. A DNA fragment having the base sequence of the translated region in SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159, which is human cDNA encoding the protein of claim 1.

4. The DNA fragment of claim 3, which consists of any one of the base sequences of SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157 or 159.

5. An expression vector, which is capable of expressing any one of the DNA fragments of claims 2 to 4 by in vitro translation or within host cell.

6. The expression vector of claim 5, which is capable of expressing a fused DNA fragment of the DNA fragment of any one of claims 2 to 4 and the DNA fragment encoding a fluorescent protein.

7. A fluorescent protein-fused protein, which is an expression product of the expression vector of claim 6.

8. A cell transformed with the expression vector of claim 5 or 6, which is capable of producing the protein of claim 1 or the fluorescent protein-fused protein of claim 7.

9. An antibody against the protein of claim 1.
